SEQUENCE LISTING (64 sequences)

SEQ ID NO: 1 - DNA, 863 bp, Homo sapiens
caaggttcagagtcacccatctcagcaagcccagaagtatctgcaatatctacgatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacatctggtccaacatgaaaacaattcttattgactcatacaccaggtcacgctttcatgaattctgtcatttcaaagactctcacccctgctataactatgaccatgctgataaactgatttatctatttaaatatttatttaactattcataagatttaaattatttttgttcatataacgtcatgtgcacctttacactgtggttagtgtaataaaacatgttccttatatttactc

SEQ ID NO: 2 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 3 - DNA, 1143 bp, Homo sapiens
gagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatatgttctttgtatttggtaaatttattttgtgttgttcattgaacttttgctatggaaacttttgtacttgtttattctttaaaatgaaattccaagcctaattgtgcaacctgattacagaataactggtacacttcatttatccatcaatattatattcaagatataagtaaaaataaactttctgtaaacca

SEQ ID NO: 4 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 5 - DNA, 982 bp, Homo sapiens
agaaaacctagaggccgaagttcaaggttatccatctcaagtagcctagcaatatttgcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcatttctcctgcctgaaggacagacatgatttcggattccccgaggaggagtttgatggccaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaacagagaagaaatacagcccttgtgcctgggaggttgtcagagcagaaatcatgagatccctctcgttttcaacaaacttgcaaaaaagattaaggaggaaggattgaaacctggttcaacatggaaatgatcctgattgactaatacattatctcacactttcatgagttcttccatttcaaagactcacttctataaccaccacgagttgaatcaaaattttcaaatgttttcagcagtgtgaagaagcttggtgtatacctgtgcaggcactagtcctttacagatgacaatgctgatgtctctgttcatctatttatttaaatatttatttatttttaaaatttaaattattttttatgtgatatcatgagtacctttacattgtggtgaatgtaacaatatatgttcttcatatttagccaatatattaatttcctttttcattaaa

SEQ ID NO: 6 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 7 - DNA, 700 bp, Homo sapiens
gcccaaggttcagggtcactcaatctcaacagcccagaagcatctgcaacctccccaatggccttgccctttgttttactgatggccctggtggtgctcaactgcaagtcaatctgttctctgggctgtgatctgcctcagacccacagcctgagtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgagaaaatactttcaaagaatcaccctctatctgacagagaagaaatacagcccttgtgcatgggaggttgtcagagcagaaatcatgagatccttctctttatcagcaaacttgcaagaaagattaaggaggaaggaatgaaaactggttcaacatcgaaatgattctcattgactagtacaccatttcacacttcttgagttctgccgtttca

SEQ ID NO: 8 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 9 - DNA, 570 bp, Homo sapiens
atggctttgccttttgctttactgatggccctggtggtgctcagctgcaagtcaagctgctctctggactgtgatctgcctcagacccacagcctgggtcacaggaggaccatgatgctcctggcacaaatgaggagaatctctcttttctcctgtctgaaggacagacatgacttcagatttccccaggaggagtttgatggcaaccagttccagaaggctgaagccatctctgtcctccatgaggtgattcagcagaccttcaacctcttcagcacaaaggactcatctgttgcttgggatgagaggcttctagacaaactctatactgaactttaccagcagctgaatgacctggaagcctgtgtgatgcaggaggtgtgggtgggagggactcccctgatgaatgaggactccatcctggctgtgagaaaatacttccaaagaatcactctctacctgacagagaaaaagtacagcccttgtgcctgggaggttgtcagagcagaaatcatgagatccttctcttcatcaagaaacttgcaagaaaggttaaggaggaaggaataa

SEQ ID NO: 10 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 11 - DNA, 736 bp, Homo sapiens
tacccacctcaggtagcctagtgatatttgcaaaatcccaatggcccggtccttttctttactgatggtcgtgctggtactcagctacaaatccatctgctctctgggctgtgatctgcctcagacccacagcctgcgtaataggagggccttgatactcctggcacaaatgggaagaatctctcctttctcctgcttgaaggacagacatgaattcagattcccagaggaggagtttgatggccaccagttccagaagactcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggacttcatcctggctgtgaggaaatacttccaaagaatcactctttatctaatggagaagaaatacagcccttgtgcctgggaggttgtcagagcagaaatcatgagatccttctctttttcaacaaacttgaaaaaaggattaaggaggaaggattgaaaactggttcatcatggaaatgattctcattgactaatgcatcatctcacactttcatgagttcttccatttcaaagactcacttctataaccaccacaagttaatcaaaatttccaaatgttttc

SEQ ID NO: 12 - protein, 189 aa, Homo sapiens
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 13 - DNA, 1039 bp, Homo sapiens
accagctcagcagcatccacaacatctacaatggccttgactttttatttactggtggccctagtggtgctcagctacaagtcattcagctctctgggctgtgatctgcctcagactcacagcctgggtaacaggagggccttgatactcctggcacaaatgcgaagaatctctcctttctcctgcctgaaggacagacatgactttgaattcccccaggaggagtttgatgataaacagttccagaaggctcaagccatctctgtcctccatgagatgatccagcagaccttcaacctcttcagcacaaaggactcatctgctgctttggatgagacccttctagatgaattctacatcgaacttgaccagcagctgaatgacctggagtcctgtgtgatgcaggaagtgggggtgatagagtctcccctgatgtacgaggactccatcctggctgtgaggaaatacttccaaagaatcactctatatctgacagagaagaaatacagctcttgtgcctgggaggttgtcagagcagaaatcatgagatccttctctttatcaatcaacttgcaaaaaagattgaagagtaaggaatgagacctggtacaacacggaaatgattcttatagactaatacagcagctcacacttcgacaagttgtgctctttcaaagacccttgtttctgccaaaaccatgctatgaattgaatcaaatgtgtcaagtgttttcaggagtgttaagcaacatcctgttcagctgtatgggcactagtcccttacagatgaccatgctgatggatctattcatctatttatttaaatctttatttagttaactatctatagggcttaaattagttttgttcatattatattatgtgaacttttacattgtgaattgtgtaacaaaaacatgttctttatatttattattttgccttgtttattaaatttttactatagaaaaattctttatttattctttaaaattgaactccaaccctgattgtgcaaactgattaaagaatggatggt

SEQ ID NO: 14 - protein, 189 aa, Homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 15 - DNA, 963 bp, Homo sapiens
caaggttatccatctcaagtagcctagcaatatttgcaacatcccaatggccctgtccttttctttacttatggccgtgctggtgctcagctacaaatccatctgttctctgggctgtgatctgcctcagacccacagcctcggtaataggagggccttgatactcctgggacaaatgggaagaatctctcctttctcctgcctgaaggacagacatgatttccgaatcccccaggaggagtttgatggcaaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaatagagaggaaatacagcccttgtgcctgggaggttgtcagagcagaaatcatgagatccctctcgttttcaacaaacttgcaaaaaagattaaggaggaaggattgaaaactggttcaacatggcaatgatcctgattgactaatacattatctcacactttcatgagttcttccatttcaaagactcacttctataaccacgacgcgttgaatcaaaattttcaaatgttttcagcagtgtaaagaagtgtcgtgtatacctgtgcaggcactagtcctttacagatgaccattctgatgtctctgttcatcttttgtttaaatatttatttaattatttttaaaatttatgtaatatcatgagtcgctttacattgtggttaatgtaacaatatatgttcttcatatttagccaatatattaatttcctttttcattaaatttttactatac

SEQ ID NO: 16 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 17 - DNA, 705 bp, Homo sapiens
agagaacctagagcccaaggttcagagtcacccatctcagcaagcccagaagcatctgcaatatctatgatggcctcgccctttgctttactgatggccctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacacctggtccaacatgaaacaattcttattgactcatataccaggtcacgctttcatgaattctgc

SEQ ID NO: 18 - protein, 190 aa, Homo sapiens
MMASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 19 - DNA, 778 bp, Homo sapiens
gttacccctcatcaaccagcccagcagcatcttcgggattcccaatggcattgccctttgctttaatgatggccctggtggtgctcagctgcaagtcaagctgctctctgggctgtaatctgtctcaaacccacagcctgaataacaggaggactttgatgctcatggcacaaatgaggagaatctctcctttctcctgcctgaaggacagacatgactttgaatttccccaggaggaatttgatggcaaccagttccagaaagctcaagccatctctgtcctccatgagatgatgcagcagaccttcaatctcttcagcacaaagaactcatctgctgcttgggatgagaccctcctagaaaaattctacattgaacttttccagcaaatgaatgacctggaagcctgtgtgatacaggaggttggggtggaagagactcccctgatgaatgaggactccatcctggctgtgaagaaatacttccaaagaatcactctttatctgatggagaagaaatacagcccttgtgcctgggaggttgtcagagcagaaatcatgagatccctctctttttcaacaaacttgcaaaaaagattaaggaggaaggattgaaaactggttcatcatggaaatgattctcattgactaatacatcatctcacactttcatgagttcttccatttcaaagactcacttctcctataaccaccacaagttgaatcaaaattttcaaatgttttcaggagtgtaaagaagcatcatgtatacctgtgca

SEQ ID NO: 20 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 21 - DNA, 939 bp, Homo sapiens
atcccaatggccctgtccttttctttactgatggccgtgctggtgctcagctacaaatccatctgttctctgggctgtgatctgcctcagactcacagcctgggtaataggagggccttgatactcctggcacaaatgggaagaatctctcatttctcctgcctgaaggacagatatgatttcggattcccccaggaggtgtttgatggcaaccagttccagaaggctcaagccatctctgccttccatgagatgatccagcagaccttcaatctcttcagcacaaaggattcatctgctgcttgggatgagaccctcctagacaaattctacattgaacttttccagcaactgaatgacctagaagcctgtgtgacacaggaggttggggtggaagagattgccctgatgaatgaggactccatcctggctgtgaggaaatactttcaaagaatcactctttatctgatggggaagaaatacagcccttgtgcctgggaggttgtcagagcagaaatcatgagatccttctctttttcaacaaacttgcaaaaaggattaagaaggaaggattgaaaactcattcaacatggaaatgatcctcattgattaatacatcatctcacactttcatgagttcttccatttcaaagactcacttctataaccaccacaagttgaatcaaaatttcaaaatgttttcaggagtgtaaagaagcatcgtgtttacctgtgcaggcactagtcctttacagatgaccatgctgatgtctctattcatctatttatttaaatatttatttatttaactatttttaaggtttaaatcatgttttatgtaatatcatgtgtacctttacattttgcttaatgtaacaatatatgttcttcatatttagttaatatattaacttccttttcattaaatttttactatac

SEQ ID NO: 22 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 23 - DNA, 980 bp, Homo sapiens
gttcaaggttacccatctcaagtagcctagcaacatttgcaacatcccaatggccctgtccttttctttactgatggccgtgctggtgctcagctacaaatccatctgttctctaggctgtgatctgcctcagacccacagcctgggtaataggagggccttgatactcctggcacaaatgggaagaatctctcctttctcctgcctgaaggacagacatgactttggacttccccaggaggagtttgatggcaaccagttccagaagactcaagccatctctgtcctccatgagatgatccagcagaccttcaatctcttcagcacagaggactcatctgctgcttgggaacagagcctcctagaaaaattttccactgaactttaccagcaactgaataacctggaagcatgtgtgatacaggaggttgggatggaagagactcccctgatgaatgaggactccatcctggctgtgaggaaatacttccaaagaatcactctttatctaacagagaagaaatacagcccttgtgcctgggaggttgtcagagcagaaatcatgagatctctctctttttcaacaaacttgcaaaaaatattaaggaggaaggattgaaaactggttcaacatggcaatgatcctgattgactaatacattatctcacactttcatgagttcctccatttcaaagactcacttctataaccaccacgagttgaatcaaaattttcaaatgttttcagcagtgtaaagaagcgtcgtgtatacctgtgcaggcactagtactttacagatgaccatgctgatgtctctgttcatctatttatttaaatatttatttaattatttttaagatttaaattatttttttatgtaatatcatgtgtacctttacattgtggtgaatgtaacaatatatgttcttcatatttagccaatatattaatttcctttttcattaaatttttactatac

SEQ ID NO: 24 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 25 - DNA, 1024 bp, Homo sapiens
ttcaaggttacccatctcaagtagcctagcaatattggcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggactccatcctggctgtgaagaaatacttccaaagaatcactctttatctgacagagaagaaatacagcccttgtgcctgggaggttgtcagagcagaaatcatgagatccttctctttatcaaaaatttttcaagaaagattaaggaggaaggaatgaaacctgtttcaacatggaaatgatctgtattgactaatacaccagtccacacttctatgacttctgccatttcaaagactcatttctcctataaccaccgcatgagttgaatcaaaattttcagatcttttcaggagtgtaaggaaacatcatgtttacctgtgcaggcactagtcctttacagatgaccatgctgatagatctaattatctatctattgaaatatttatttatttattagatttaaattatttttgtccatgtaatattatgtgtacttttacattgtgttatatcaaaatatgttatttatatttagtcaatatattattttctttttattaatttttactattaaaacttcttatattatttgtttattctttaataaagaaataccaagccc

SEQ ID NO: 26 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 27 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 28 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 29 - DNA, 1514 bp, Homo sapiens
gatctggtaaacctgaagcaaatatagaaacctatagggcctgacttcctacataaagtaaggagggtaaaaatggaggctagaataagggttaaaattttgcttctagaacagagaaaatgatttttttcatatatatatgaatatatattatatatacacatatatacatatattcactatagtgtgtatacataaatatataatatatatattgttagtgtagtgtgtgtctgattatttacatgcatatagtatatacacttatgactttagtacccagacgtttttcatttgattaagcattcatttgtattgacacagctgaagtttactggagtttagctgaagtctaatgcaaaattaatagattgttgtcatcctcttaaggtcatagggagaacacacaaatgaaaacagtaaaagaaactgaaagtacagagaaatgttcagaaaatgaaaaccatgtgtttcctattaaaagccatgcatacaagcaatgtcttcagaaaacctagggtccaaggttaagccatatcccagctcagtaaagccaggagcatcctcatttcccaatggccctcctgttccctctactggcagccctagtgatgaccagctatagccctgttggatctctgggctgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttgaaatgattctcattgattaatttgccatataacacttgcacatgtgactctggtcaattcaaaagactcttatttcggctttaatcacagaattgactgaattagttctgcaaatactttgtcggtatattaagccagtatatgttaaaaagacttaggttcaggggcatcagtccctaagatgttatttatttttactcatttatttattcttacattttatcatatttatactatttatattcttatataacaaatgtttgcctttacattgtattaagataacaaaacatgttcagctttccatttggttaaatattgtattttgttatttattaaattattttcaaac

SEQ ID NO: 30 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 31 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 32 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 33 - DNA, 3539 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccgacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccactttatgtgcatcttatttctgtcaacttgtattttttttcttgtatttttccaattagctcctcctttttccttccagtctaaaaaaggaatcctctgtgtcttcaaagcaaagctctttactttccccttggttctcataactctgtgatcttgctctcggtgcttccaactcatccacgtcctgtctgtttcctctgtatacaaaaccctttctgcccctgctgacacagacatcctctatgccagcagccagccaaccctttcattagaacttcaagctctccaaaggctcagattataactgttgtcatatttatatgaggctgttgtcttttccttctgagcctgcctttctcccccccacccaggagtatcctcttgccaaatcaaaagactttttccttgggctttagccttaaagatacttgaaggtctaggtgctttaacctcacataccctcacttaaacttttatcactgttgcatataccagttgtgatacaataaagaatgtatctggattttgtgcctagttcctagcacacagcttcaaaaattctagagtttcctgataggagtgtcttttgtattcataacaagcccttttcacccatgcctgggtttatgctaacaaggttacccatggtgggcccttagtttcaaggaaggagttggccaagccagaaagaccaagcatgtggttaaagcattggaattttcagccccatcccacccccaatctccaaggaggtgatggggctggaaattgagttcaattttaacatggccagtgatttaagcaatgctgcctatgtaaagaaaccccaataaaaactctggacagtgaggcttggggagcttcctgattggcagacattccaatgtactaggaaggtagcgcatcttgattccacagggacaaaggctcctgagctctgggcccttccagtgcttgccaccctacatactctttgtctggctcttcatttgtattctttataataaaatggtgattgtaagtagagca

SEQ ID NO: 34 - DNA, 3647 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctgcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccggggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagccccctaagcccccactacaagtgatcctcaggcaggtaaccccagattcatgcactgtagggtgctgcgcagcatccctagtctctacccagtagatgccactagccctcctctcccagtgacaaccaaaagtcttcagacattgtcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaaatgtcatgtggctacctgtaacttgaaggtggctacaaagatgactgtggacgtgggttgcactggccacccaaggatgtctgccacacctctccaaagccctccctacctaccaagatatacctgatatattccaccaggatatcctccctccagatatacttggttctctccaccaggttctttctttaaagcaggatttctcaactttgatacttactcacatttggggctagacagttctttgtttggaggctctcttgtgcattgtaggatgttgagcagcatctctggcctgtacccagtagatgccacccagttgtgacaattaaaagtgtcttgagactttatcatgtgtcttctgccctaggtgagaacccttgcactagaggaaccctacaccccaaccctggggggaatgtagggaagaggtggccaagccaaccgtggggttagctctaattattaagatatgcattataaataaataccaaaaaattgtctctggcaatagttaccttcccagatacaggtccccccttttttcccctaactcttttaagcaatgattgtaactattaggagacattgctctcccacgtatgtttttctttttagacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccacttt

SEQ ID NO: 35 - DNA, 2123 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagtaagcattgctgggtgtcaggagagaaaagccaaagaagcgggtgccagacagctctgtgcaacctctaggccatgagtgggatagataccactgctgctttaaaaaatgggagaccatagaccctcaggagagaagaatcccttctaccctggactcgctctcttctctggaactaacttctcccccataccctgattgtctttggagaaaatgttctggattctagaatctaaggcagagccttttaagccatactgtacacataaatcacctggaaccttgttaaaatgcagatcctgactcaggaggtctgagttagagcccaggatttcatatttctagccagctccatgatgagctgctggtccgcagatcatgcttgcaggttttgaccagagtcagtgttggttagagtaagaggatgaggcagacatctgggaaaagtccagctggggcaagcatttgaagtctgccttcctaccaggtcaaaatcaaggcaacgaccttccatagataactatcaaagcttgagggggtgccttgaacccaactcctaaatccctaagacctgcccacctcttgtgtctcctgtctcagcaaacattcccacactcttgcatattgttaaagtaacctctgcttaccaggcttctggtttaataaaagatggctagagtgactccatcttaaagcaagtagctaggcactcaaaaggaacctacaggcttaatacttgggtctgaaaatagccacagtctaagctgaccaccaattataattgcagaatatttaaggccatacaaaacatctcccactaagcctacaaaatgtccaggtgtcctaaaagttcagcccacttaaaggcagcattaatgagcaggtttaggttgaaggattaatggtcatcaataccactgttaagaagaaaattcttggccaaattgaatttaatggagtttaactgagcagacaattcacaaatctagaagcctcctgagccagagtaggttcagagagtcttgaacacagccacgtggtggaagaagatttatggacaggaaaaggaaaatgatgtactgaaaatgaaagtgaggtacagaaacagccagactggttatagctcagcattggccttatttgaacgagatttgaacagttggccacctttgattggccgaaactcagtgattggcacaagagtaggttgcagtctgtttacacatccttttaggttatagttcaccatgtacagagaaattttaggccaaacttaaaatatgtaaggaggcagctttaggctaaacttgatttaacagcaccaataccccctacctttagtgagcacatctgcacattccaattttaatgacagctccttagaatttcttatcaacgaagacactaacaaagaatggcgcattcctccttctcctttctgaggatgccctaccctgtaacaaagtcgtttctaataaatttgcttctttcaccataaaaaaaaaaaaaaaaaaaaaaaaaaaaaaaaaaaaaaa

SEQ ID NO: 36 - protein, 719 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 37 - protein, 687 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPVKVI

SEQ ID NO: 38 - protein, 172 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSKHCWVSGEKSQRSGCQTALCNL

SEQ ID NO: 39 - DNA, 3520 bp, Homo sapiens
tcagtagctgaggctgcggttccccgacgccacgcagctgcgcgcagctggttcccgctctgcagcgcaacgcctgaggcagtgggcgcgctcagtcccgggaccaggcgttctctcctctcgcctctgggcctgggaccccgcaaagcggcgatggagcggaggtcgcggaggaagtcgcggcgcaacgggcgctcgaccgcgggcaaggccgccgcgacccagcccgcgaagtctccgggcgcacagctctggctctttcccagcgccgcgggcctccaccgcgcgctgctccggagggtggaggtgacgcgccaactctgctgctcgccggggcgcctcgcggtcttggaacgcggcggggcgggcgtccaggttcaccagctgctcgccgggagcggcggcgcccggacgccgaaatgcattaaattaggaaaaaacatgaagatacattccgtggaccaaggagcagagcacatgctgattctctcatcagatggaaaaccatttgagtatgacaactatagcatgaaacatctaaggtttgaaagcattttacaagaaaaaaaaataattcagatcacatgtggagattaccattctcttgcactctcaaaaggtggtgagctttttgcctggggacagaacctgcatgggcagcttggagttggaaggaaatttccctcaaccaccacaccacagattgtggagcacctcgcaggagtacccttggctcagatttctgccggagaagcccacagcatggccttatccatgtctggcaacatttattcatggggaaaaaatgaatgtggacaactaggcctgggccacactgagagtaaagatgatccatcccttattgaaggactagacaatcagaaagttgaatttgtcgcttgtggtggctctcacagtgccctactcacacaggatgggctgctgtttactttcggtgctggaaaacatgggcaacttggtcataattcaacacagaatgagctaagaccctgtttggtggctgagcttgttgggtatagagtgactcagatagcatgtggaaggtggcacacacttgcctatgtttctgatttgggaaaggtcttttcctttggttctggaaaagatggacaactgggaaatggtggaacacgtgaccagctgatgccgcttccagtgaaagtatcatcaagtgaagaactcaaacttgaaagccatacctcagaaaaggagttaataatgattgctggagggaatcaaagcattttgctctggataaagaaagagaattcatatgttaatctgaagaggacaattcctactctgaatgaagggactgtaaagagatggattgctgatgtggagactaaacggtggcagagcacaaaaagggaaatccaagagatattttcatctcctgcttgtctaactggaagttttttaaggaaaagaagaactacagaaatgatgcctgtttatttggacttaaataaagcaagaaacatcttcaaggagttaacccaaaaggactggattactaacatgataaccacctgcctcaaagataatctgctcaaaagacttccatttcattctccaccccaagaagctttagaaattttcttccttctcccagaatgtcctatgatgcatatttccaacaactgggagagccttgtggttccatttgcaaaggttgtttgtaaaatgagtgaccagtcttcactggttctggaagagtattgggcaactctgcaagaatccactttcagcaaactggtccagatgtttaaaacagccgtcatatgccagttggattactgggatgaaagtgctgaggagaatggtaatgttcaagctctcctagaaatgttgaagaagctgcacagggtaaaccaggtgaaatgtcaactacctgaaagtattttccaagtagacgaactcttgcaccgtctcaatttttttgtagaagtatgcagaaggtacttgtggaaaatgactgtggacgcttcagaaaatgtacaatgctgcgtcatattcagtcactttccatttatctttaataatctgtcgaaaattaaactactacatacagacacacttttaaaaatagagagtaaaaaacataaagcttatcttaggtcggcagcaattgaggaagaaagagagtctgaattcgctttgaggcccacgtttgatctaacagtcagaaggaatcacttgattgaggatgttttgaatcagctaagtcaatttgagaatgaagacctgaggaaagagttatgggtttcatttagtggagaaattgggtatgacctcggaggagtcaagaaagagttcttctactgtctgtttgcagagatgatccagccggaatatgggatgttcatgtatcctgaaggggcttcctgcatgtggtttcctgtcaagcctaaatttgagaagaaaagatacttcttttttggggttctatgtggactttccctgttcaattgcaatgttgccaaccttcctttcccactggcactgtttaagaaacttttggaccaaatgccatcattggaagacttgaaagaactcagtcctgatttgggaaagaatttgcaaacacttctggatgatgaaggtgataactttgaggaagtattttacatccattttaatgtgcactgggacagaaacgacacaaacttaattcctaatggaagtagcataactgtcaaccagactaacaagagagactatgtttctaagtatatcaattacattttcaacgactctgtaaaggcggtttatgaagaatttcggagaggattttataaaatgtgcgacgaagacattatcaaattattccaccccgaagaactgaaggatgtgattgttggaaatacagattatgattggaaaacatttgaaaagaatgcacgttatgaaccaggatataacagttcacatcccaccatagtgatgttttggaaggctttccacaaattgactctggaagaaaagaaaaaattccttgtatttcttacaggaactgacagactacaaatgaaagatttaaataatatgaaaataacattttgctgtcctgaaagttggaatgaaagagaccctataagagcactgacatgtttcagtgtcctcttcctccctaaatattctacaatggaaacagttgaagaagcgcttcaagaagccatcaacaacaacagaggatttggctgaccagcttgcttgtccaacagccttattttgttgttgttatcgttgttgttgttgttgttgttgttgtttctctactttgttttgttttaggcttttagcagcctgaagccatggtttttcatttctgtctctagtgataagcaggaaagagggatgaagaagagggtttactggccggttagaacccgtgactgtattctctcccttggatacccctatgcctacatcatattccttacctcttttgggaaatatttttcaaaaataaaataaccgaaaaattaacataaaa

SEQ ID NO: 40 - protein, 1024 aa, Homo sapiens
MERRSRRKSRRNGRSTAGKAAATQPAKSPGAQLWLFPSAAGLHRALLRRVEVTRQLCCSPGRLAVLERGGAGVQVHQLLAGSGGARTPKCIKLGKNMKIHSVDQGAEHMLILSSDGKPFEYDNYSMKHLRFESILQEKKIIQITCGDYHSLALSKGGELFAWGQNLHGQLGVGRKFPSTTTPQIVEHLAGVPLAQISAGEAHSMALSMSGNIYSWGKNECGQLGLGHTESKDDPSLIEGLDNQKVEFVACGGSHSALLTQDGLLFTFGAGKHGQLGHNSTQNELRPCLVAELVGYRVTQIACGRWHTLAYVSDLGKVFSFGSGKDGQLGNGGTRDQLMPLPVKVSSSEELKLESHTSEKELIMIAGGNQSILLWIKKENSYVNLKRTIPTLNEGTVKRWIADVETKRWQSTKREIQEIFSSPACLTGSFLRKRRTTEMMPVYLDLNKARNIFKELTQKDWITNMITTCLKDNLLKRLPFHSPPQEALEIFFLLPECPMMHISNNWESLVVPFAKVVCKMSDQSSLVLEEYWATLQESTFSKLVQMFKTAVICQLDYWDESAEENGNVQALLEMLKKLHRVNQVKCQLPESIFQVDELLHRLNFFVEVCRRYLWKMTVDASENVQCCVIFSHFPFIFNNLSKIKLLHTDTLLKIESKKHKAYLRSAAIEEERESEFALRPTFDLTVRRNHLIEDVLNQLSQFENEDLRKELWVSFSGEIGYDLGGVKKEFFYCLFAEMIQPEYGMFMYPEGASCMWFPVKPKFEKKRYFFFGVLCGLSLFNCNVANLPFPLALFKKLLDQMPSLEDLKELSPDLGKNLQTLLDDEGDNFEEVFYIHFNVHWDRNDTNLIPNGSSITVNQTNKRDYVSKYINYIFNDSVKAVYEEFRRGFYKMCDEDIIKLFHPEELKDVIVGNTDYDWKTFEKNARYEPGYNSSHPTIVMFWKAFHKLTLEEKKKFLVFLTGTDRLQMKDLNNMKITFCCPESWNERDPIRALTCFSVLFLPKYSTMETVEEALQEAINNNRGFG

SEQ ID NO: 41 - DNA, 2037 bp, Homo sapiens
gggaagctcgggccggcagggtttccccgcacgctggcgcccagctcccggcgcggaggccgctgtaagtttcgctttccattcagtggaaaacgaaagctgggcggggtgccacgagcgcggggccagaccaaggcgggcccggagcggaacttcggtcccagctcggtccccggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgagccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgatcatctcttctccataagatagtgtgataaacacagtcatgaataaagttattttccacaaaa

SEQ ID NO: 42 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC

SEQ ID NO: 43 - DNA, 1642 bp, Homo sapiens
aagccacggggagaaacgttgcagcccgcgccgaacgccgggcagcacaaaggatccccgactgccggggagcggtgctcggagggcacaggtctacgccatcccccacgcagtttcggagatggagcgctgggcccatggagggaaggcggcaggctcggcggctccggcagcttgctggggcaggggctcaaggcggcagtccgatagtggaggccgctgagaactgtcacggagctgcgtctgtacagcgagcatcccttatttattcagggcgagtgtgtatttggggcggcgtgcagggggctgacaaagaccggagagctcccggtgcggccgccggcggagcgaagactggaacccgtatgagcgccccccagcgcccctgagcgctcgccgccggtgcacggcgcaccccgcgggaggcagggatcagcaaagccgtgcgccccgaggcccgcccccgtctccgcacaaagaccgagctggaggatcttcagaagaagcctcccccatacctgcggaacctgtccagcgatgatgccaatgtcctggtgtggcacgctctcctcctacccgaccaacctccctaccacctgaaagccttcaacctgcgcatcagcttcccgccggagtatccgttcaagcctcccatgatcaaattcacaaccaagatctaccaccccaacgtggacgagaacggacagatttgcctgcccatcatcagcagtgagaactggaagccttgcaccaagacttgccaagtcctggaggccctcaatgtgctggtgaatagaccgaatatcagggagcccctgcggatggacctcgctgacctgctgacacagaatccggagctgttcagaaagaatgccgaagagttcaccctccgattcggagtggaccggccctcctaactcatgttctgaccctctgtgcactggatcctcggcatagcggacggacacacctcatggactgaggccagagccccctgtggcccattccccattcatttttcccttcttaggttgttagtcattagtttgtgtgtgtgtgtggtggagggaagggagctatgagtgtgtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccatttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccattaaagtcagagatgaattctggaaaaaaaaaaaaaaaaaaaaaaa

SEQ ID NO: 44 - DNA, 1283 bp, Homo sapiens
gggggtggggtccccggggcggggcggggcgcgctgtgtcgcgggtcggagctcggtcctgctggaggccacgggtgccacacactcggtcccgacatgatggcgagcatgcgagtggtgaaggagctggaggatcttcagaagaagcctcccccatacctgcggaacctgtccagcgatgatgccaatgtcctggtgtggcacgctctcctcctacccgaccaacctccctaccacctgaaagccttcaacctgcgcatcagcttcccgccggagtatccgttcaagcctcccatgatcaaattcacaaccaagatctaccaccccaacgtggacgagaacggacagatttgcctgcccatcatcagcagtgagaactggaagccttgcaccaagacttgccaagtcctggaggccctcaatgtgctggtgaatagaccgaatatcagggagcccctgcggatggacctcgctgacctgctgacacagaatccggagctgttcagaaagaatgccgaagagttcaccctccgattcggagtggaccggccctcctaactcatgttctgaccctctgtgcactggatcctcggcatagcggacggacacacctcatggactgaggccagagccccctgtggcccattccccattcatttttcccttcttaggttgttagtcattagtttgtgtgtgtgtgtggtggagggaagggagctatgagtgtgtgtgttgtgtatggactcactcccaggttcacctggccacaggtgcacccttcccacaccctttacattccccagagccaagggagtttaagtttgcagttacaggccagttctccagctctccatcttagagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccatttattttccacggatgagtcacaatctgaagaatcaaacttccatcctgaaaatctatatgtttcaaaaccacttgccatcctgttagattgccagttcctgggaccaggcctcagactgtgaagtatatatcctccagcattcagtccagggggagccacggaaaccatgttcttgcttaagccattaaagtcagagatgaattctggaaaaaaaaaaaaaaaaaaaaaaa

SEQ ID NO: 45 - protein, 87 aa, Homo sapiens
MIKFTTKIYHPNVDENGQICLPIISSENWKPCTKTCQVLEALNVLVNRPNIREPLRMDLADLLTQNPELFRKNAEEFTLRFGVDRPS

SEQ ID NO: 46 - protein, 153 aa, Homo sapiens
MMASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPS

SEQ ID NO: 47 - DNA, 685 bp, Homo sapiens
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 48 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 49 - DNA, 5889 bp, Homo sapiens
gctgccagctgagtttttttgctgctttgagtctcagttttctttctttcctagagtctctgaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaagctgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacatgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgatatcaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaactatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgcaaaaaaaaaaaaaaaaaataagaaagaacaaaacaaaccccacaaaaattagctgggtatgatggcacgtgcctgtagtcccagttactcaggatgattgattgagccttggaggtggaggctacagtgagctgagattgtgccactgtactctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaaataaatattcctcttctaacaaacccatgcttgattttcattaattgaattccaaatcatcctagccatgtgtccttccatttaggttactggggcaaatcagtaagaaagttcttatatttatgctccaaataattctgaagtcctcttactagctgtgaaagctagtactattaagaaagaaaacaaaattcccaaaagatagctttcacttttttttttccttaaagacttcctaattctcttctccaaattcttagtcttcttcaaaataatatgctttggttcaatagttatccacattctgacagtctaatttagttttaatcagaattatactcatcttttgggtagtcatagatattaagaaagcaagagtttcttatgtccagttatggaatatttcctaaagcaaggctgcaggtgaagttgtgctcaagtgaatgttcaggagacacaattcagtggaagaaattaagtctttaaaaaagacctaggaataggagaaccatggaaattgaggaggtaggcctacaagtagatattgggaacaaaattagagaggcaaccagaaaaagttattttaggctcaccagagttgttcttattgcacagtaacacaccaatataccaaaacagcaggtattgcagtagagaaagagtttaataattgaatggcagaaaaatgaggaaggttgaggaaacctcaaatctacctccctgctgagtctaagtttaggatttttaagagaaaggcaggtaaggtgctgaaggtctggagctgctgatttgttggggtatagggaatgaaatgaaacatacagagatgaaaactggaagtttttttttgtttgttttgttttttttttgttgttgtttttttttttttttgtttttttgctgagtcaattccttggagggggtcttcagactgactggtgtcagcagacccatgggattccaagatctggaaaactttttagatagaaacttgatgtttcttaacgttacatatattatcttatagaaataactaagggaagttagtgccttgtgaccacatctatgtgacttttaggcagtaagaaactataaggaaaggagctaacagtcatgctgtaagtagctacagggaattggcttaaagggcaagttggttagtacttagctgtgtttttattcaaagtctacattttatgtagtggttaatgtttgctgttcattaggatggtttcacagttaccatacaaatgtagaagcaacaggtccaaaaagtagggcatgattttctccatgtaatccagggagaaaacaagccatgaccattgttggttgggagactgaaggtgattgaaggttcaccatcatcctcaccaacttttgggccataattcacccaaccctttggtggagcctgaaaaaaatctgggcagaatgtaggacttctttattttgtttaaaggggtaacacagagtgcccttatgaaggagttggagatcctgcaaggaagagaaggagtgaaggagagatcaagagagagaaacaatgaggaacatttcatttgacccaacatcctttaggagcataaatgttgacactaagttatcccttttgtgctaaaatggacagtattggcaaaatgataccacaacttcttattctctggctctatattgctttggaaacacttaaacatcaaatggagttaaatacatatttgaaatttaggttaggaaatattggtgaggaggcctcaaaaagggggaaacatcttttgtctgggaggatattttccattttgtggatttccctgatctttttctaccaccctgaggggtggtgggaattatcattttgctacattttagaggtcatccaggatttttgaaactttacattctttacggttaagcaagatgtacagctcagtcaaagacactaaattcttcttagaaaaatagtgctaaggagtatagcagatgacctatatgtgtgttggctgggagaatatcatcttaaagtgagagtgatgttgtggagacagttgaaatgtcaatgctagagcctctgtggtgtgaatgggcacgttaggttgttgcattagaaagtgactgtttctgacagaaatttgtagctttgtgcaaactcacccaccatctacctcaataaaatatagagaaaagaaaaatagagcagtttgagttctatgaggtatgcaggcccagagagacataagtatgttcctttagtcttgcttcctgtgtgccacactgcccctccacaaccatagctgggggcaattgtttaaagtcattttgttcccgactagctgccttgcacattatcttcattttcctggaatttgatacagagagcaatttatagccaattgatagcttatgctgtttcaatgtaaattcgtggtaaataacttaggaactgcctcttctttttctttgaaaacctacttataactgttgctaataagaatgtgtattgttcaggacaacttgtctccatacagttgggttgtaaccctcatgcttggcccaaataaactctctacttatatcagtttttcctacacttcttccttttaggtcaacaataccaagaggggttactgtgctgggtaatgtgtaaacttgtgtcttgtttagaaagataaatttaaagactatcacattgctttttcataaaacaagacaggtctacaattaatttattttgacgcaaattgataggggggccaagtaagccccatatgcttaatgatcagctgatgaataatcatctcctagcaacataactcaatctaatgctaaggtacccacaagatggcaaggctgatcaaagtcgtcatggaatcctgcaaccaaaagccatgggaatttggaagccctcaaatcccattcctaatctgatgagtctatggaccaatttgtggaggacagtagattaaatagatctgatttttgccatcaatgtaaggaggataaaaacttgcataccaattgtacacccttgcaaaatctttctctgatgttggagaaaatgggccagtgagatcatggatatagaagtacagtcaatgttcagctgtaccctcccacaatcccacttccttcctcaacacaattcaaacaaatagactcagactgtttcaggctccaggacaggaagtgcagtgtaggcaaaattgcaaaaattgagggcacaggggtggaggtgggggggttgaataacaagctgtgctaaataattacgtgtaaatatattttttcatttttaaaaattgatttcttttgcacattccatgacaatatatgtcacatttttaaaataaatgcaaagaagcatacatccaaaaaaaaaaaaaaaaa

SEQ ID NO: 50 - protein, 452 aa, Homo sapiens
MEVTTRLTWNDENHLRKLLGNVSLSLLYKSSVHGGSIEDMVERCSRQGCTITMAYIDYNMIVAFMLGNYINLHESSTEPNDSLWFSLQKKNDTTEIETLLLNTAPKIIDEQLVCRLSKTDIFIICRDNKIYLDKMITRNLKLRFYGHRQYLECEVFRVEGIKDNLDDIKRIIKAREHRNRLLADIRDYRPYADLVSEIRILLVGPVGSGKSSFFNSVKSIFHGHVTGQAVVGSDITSITERYRIYSVKDGKNGKSLPFMLCDTMGLDGAEGAGLCMDDIPHILKGCMPDRYQFNSRKPITPEHSTFITSPSLKDRIHCVAYVLDINSIDNLYSKMLAKVKQVHKEVLNCGIAYVALLTKVDDCSEVLQDNFLNMSRSMTSQSRVMNVHKMLGIPISNILMVGNYASDLELDPMKDILILSALRQMLRAADDFLEDLPLEETGAIERALQPCI

SEQ ID NO: 51 - DNA, 3512 bp, Homo sapiens
aactcagctgagtgttagtcaaagaaggtgtgtcctgctccccaatgacaggttgctcagagactgctgatttccatccctatataaagagagtccctggcatacagagactgctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggtgtggtgcagcccagcccacggtggctgttccatgaatgctggctacctatgtgtgtggtacctgttgtgtccctttctcttcaaagatcctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatctctgagaagtgacggcacactgggttggcataagatatcctaaaatcacgctggaaccttgggcaaggaagaatgtgagcaagagtagagagagtgcctggatttcatgtcagtgaagccaagtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttagctgcaatttcagtgttcctcactaggtggcatttaaatgtcgcctgatgtcattaagcaccatccaaaaagtctgcttcataatctattttcaagacttggtgattctgaaagttttggtttttgtgactttgtttctcaggaaaaaaaatattcctacttaaattttaagtctataattcaatttaaatatgtgtgtgtctcatccaggataggataggttgtcttctattttccattttacctatttactttttttgtaagaaaagagaaaaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttgggaggacaatgtgggtggatcacgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgccactgcactccagcctggatgacagagcaagactccgtctcaaaaaaaaaaaaaaaaaaaagcaagagagttcaactaagaaaggtcacatatgtgaaagcccaaggacactgtttgatatacagcaggtattcaatcagtgttatttgaaaccaaatctgaatttgaagtttgaatcttctgagttggaatgaatttttttctagctgagggaaactgtatttttctttccccaaagaggaatgtaatgtaaagtgaaataaaactataagctatgtt

SEQ ID NO: 52 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 53 - DNA, 665 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 54 - DNA, 656 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 55 - protein, 122 aa, Homo sapiens
MEASALTSSAVTSVAKVVRVASGSAVVLPLARIATVVIGGVVAMAAVPMVLSAMGFTAAGIASSSIAAKMMSAAAIANGGGVASGSLVATLQSLGATGLSGLTKFILGSIGSAIAAVIARFY

SEQ ID NO: 56 - protein, 119 aa, Homo sapiens
MEASALTSSAVTSVAKVVRVASGSAVVLPLARIATVVIGGVVAVPMVLSAMGFTAAGIASSSIAAKMMSAAAIANGGGVASGSLVATLQSLGATGLSGLTKFILGSIGSAIAAVIARFY

SEQ ID NO: 57 - DNA, 4326 bp, Homo sapiens
gctgagcgcggagccgcccggtgattggtgggggcggaagggggccgggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttctaattgctgaggtttagctgtcagttctttttgccctttgggaattcggcatggtttcattttactgcactagccaagagactttacttttaagaagtattaaaattctaaaattcaaaaaaaaaaaaaaaaaa

SEQ ID NO: 58 - DNA, 2798 bp, Homo sapiens
gctgagcgcggagccgcccggtgattggtgggggcggaagggggccgggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagtgtaagtgaacacagaagagtgacatgtttacaaacctcaagccagccttgctcctggctggggcctgttgaagatgcttgtattttacttttccattgtaattgctatcgccatcacagctgaacttgttgagatccccgtgttactgcctatcagcattttactactttaaaaaaaaaaaaaaagccaaaaaccaaatttgtatttaaggtatataaattttcccaaaactgataccctttgaaaaagtataaataaaatgagcaaaagttgat

SEQ ID NO: 59 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 60 - protein, 712 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEV

SEQ ID NO: 61 - DNA, 3330 bp, Homo sapiens
cttttcttttctccaggaagagagctgtgaccagcagcgtcccttattcgcttggccttggttcctgtttgcactggctacagcagggcactggcccctactgtcaccgccacctacacaaagaccctatctctgagcgctgcagcctactgttcagccccaggtttgaggatggatgccctggacgcttcgaagctactggatgaggagctgtattcaagacagctgtatgtgctgggctcacctgccatgcagaggattcagggagccagggtcctggtgtcaggcctgcagggcctgggggccgaggtggccaagaacttggttctgatgggtgtgggcagcctcactctgcatgatccccaccccacctgctggtccgacctggctgcccagtttctcctctcagagcaggacttggaaaggagcagagccgaggcctctcaagagctcttggctcagctcaacagagctgtccaggtcgtcgtgcacacgggtgacatcactgaggacctgctgttggacttccaggtggtggtgctgactgctgcaaagctggaggagcagctgaaggtgggcaccttgtgtcataagcatggagtttgctttctggcggctgacacccggggcctcgtggggcagttgttctgtgactttggtgaggacttcactgtgcaggaccccacagaggcagaacccctgacagctgccatccagcacatctcccagggctcccctggcattctcactctgaggaaaggggccaatacccactacttccgtgatggagacttggtgactttctcgggaattgagggaatggttgagctcaacgactgtgatccccggtctatccacgtgcgggaggatgggtccctggagattggagacacaacaactttctctcggtacttgcgtggtggggctatcactgaagtcaagagacccaagactgtgagacataagtccctggacacagccctgctccagccccatgtggtggcccagagctcccaggaagttcaccatgcccactgcctgcatcaggccttctgtgcactgcacaagttccagcacctccatggccggccaccccagccctgggatcctgttgatgcagagactgtggtgggcctggcccgggacctggaaccactgaagcggacagaggaagagccactggaagagccactggatgaggccctagtgcggacagtcgccctaagcagtgcaggtgtcttgagccctatggtggccatgctgggtgcagtagctgcccaggaagtgctgaaggcaatctccaggaagttcatgcctctggaccagtggctttactttgatgccctcgattgtcttccggaagatggggagctccttcccagtcctgaggactgtgccctgagaggcagccgctatgatgggcaaattgcagtgtttggggctggttttcaggagaaactgagacgccagcactacctcctggtgggcgctggtgccattggttgtgagctgctcaaagtctttgccctagtgggactgggggccgggaacagcgggggcttgactgttgttgacatggaccacatagagcgctccaatctcagccgtcagttcctcttcaggtcccaggacgttggtagacccaaggcagaggtggctgcagcagctgcccggggcctgaacccagacttacaggtgatcccgctcacctacccactggatcccaccacagagcacatctatggggataactttttctcccgtgtggatggtgtggctgctgccctggacagtttccaggcccggcgctatgtggctgctcgttgcacccactatctgaagccactgctggaggcaggcacatcgggcacctggggcagtgctacagtattcatgccacatgtgactgaggcctacagagcccctgcctcagctgcagcttctgaggatgccccctaccctgtctgtaccgtgcggtacttccctagcacagccgagcacaccctgcagtgggcccggcatgagtttgaagaactcttccgactgtctgcagagaccatcaaccaccaccaacaggcacacacttccctggcagacatggatgagccacagacactcaccttactgaagccagtgcttggggtcctgagagtgcgtccacagaactggcaagactgtgtggcgtgggctcttggccactggaaactctgctttcattatggcatcaaacagctgctgaggcacttcccacctaataaagtgcttgaggatggaactcccttctggtcaggtcccaaacagtgtccccagcccttggagtttgacaccaaccaagacacacacctcctctacgtactggcagctgccaacctgtatgcccagatgcatgggctgcctggctcacaggactggactgcactcagggagctgctgaagctgctgccacagcctgacccccaacagatggcccccatctttgctagtaatctagagctggcttcggcttctgctgagtttggccctgagcagcagaaggaactgaacaaagccctggaagtctggagtgtgggccctcccctgaagcctctgatgtttgagaaggatgatgacagcaacttccatgtggactttgtggtagcggcagctagcctgagatgtcagaactacgggattccaccggtcaaccgtgcccagagcaagcgaattgtgggccagattatcccagccattgccaccactacagcagctgtggcaggcctgttgggcctggagctgtataaggtggtgagtgggccacggcctcgtagtgcctttcgccacagctacctacatctggctgaaaactacctcatccgctatatgccttttgccccagccatccagacgttccatcacctgaagtggacctcttgggaccgtctgaaggtaccagctgggcagcctgagaggaccctggagtcgctgctggctcatcttcaggagcagcacgggttgagggtgaggatcctgctgcacggctcagccctgctctatgcggccggatggtcacctgaaaagcaggcccagcacctgcccctcagggtgacagaactggttcagcagctgacaggccaggcacctgctcctgggcagcgggtgttggtgctagagctgagctgtgagggtgacgacgaggacactgccttcccacctctgcactatgagctgtgacaaggcagccaccctgtcacctagctcaatggagccccggatcccaagccctgcattgtaagcccacagtaggcactcaataaatgcttgttaaaggaaggcaaaaaaaaaaaaaaaaaa

SEQ ID NO: 62 - protein, 1012 aa, Homo sapiens
MDALDASKLLDEELYSRQLYVLGSPAMQRIQGARVLVSGLQGLGAEVAKNLVLMGVGSLTLHDPHPTCWSDLAAQFLLSEQDLERSRAEASQELLAQLNRAVQVVVHTGDITEDLLLDFQVVVLTAAKLEEQLKVGTLCHKHGVCFLAADTRGLVGQLFCDFGEDFTVQDPTEAEPLTAAIQHISQGSPGILTLRKGANTHYFRDGDLVTFSGIEGMVELNDCDPRSIHVREDGSLEIGDTTTFSRYLRGGAITEVKRPKTVRHKSLDTALLQPHVVAQSSQEVHHAHCLHQAFCALHKFQHLHGRPPQPWDPVDAETVVGLARDLEPLKRTEEEPLEEPLDEALVRTVALSSAGVLSPMVAMLGAVAAQEVLKAISRKFMPLDQWLYFDALDCLPEDGELLPSPEDCALRGSRYDGQIAVFGAGFQEKLRRQHYLLVGAGAIGCELLKVFALVGLGAGNSGGLTVVDMDHIERSNLSRQFLFRSQDVGRPKAEVAAAAARGLNPDLQVIPLTYPLDPTTEHIYGDNFFSRVDGVAAALDSFQARRYVAARCTHYLKPLLEAGTSGTWGSATVFMPHVTEAYRAPASAAASEDAPYPVCTVRYFPSTAEHTLQWARHEFEELFRLSAETINHHQQAHTSLADMDEPQTLTLLKPVLGVLRVRPQNWQDCVAWALGHWKLCFHYGIKQLLRHFPPNKVLEDGTPFWSGPKQCPQPLEFDTNQDTHLLYVLAAANLYAQMHGLPGSQDWTALRELLKLLPQPDPQQMAPIFASNLELASASAEFGPEQQKELNKALEVWSVGPPLKPLMFEKDDDSNFHVDFVVAAASLRCQNYGIPPVNRAQSKRIVGQIIPAIATTTAAVAGLLGLELYKVVSGPRPRSAFRHSYLHLAENYLIRYMPFAPAIQTFHHLKWTSWDRLKVPAGQPERTLESLLAHLQEQHGLRVRILLHGSALLYAAGWSPEKQAQHLPLRVTELVQQLTGQAPAPGQRVLVLELSCEGDDEDTAFPPLHYEL

SEQ ID NO: 63 - DNA, 2808 bp, Homo sapiens
gcggcggcggcggcgcagtttgctcatactttgtgacttgcggtcacagtggcattcagctccacacttggtagaaccacaggcacgacaagcatagaaacatcctaaacaatcttcatcgaggcatcgaggtccatcccaataaaaatcaggagaccctggctatcatagaccttagtcttcgctggtatactcgctgtctgtcaaccagcggttgactttttttaagccttcttttttctcttttaccagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattcttttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaagtgatgtacaacc

SEQ ID NO: 64 - protein, 551 aa, Homo sapiens
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKSDYLSSGSFATTCESQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

(19) United States
(12) Patent Application Publication
(10) Pub. No.: US 2017/0029896 A1
(43) Pub. Date: Feb. 2, 2017

(54) PROGNOSTIC KITS, ARRAYS COMPOSITIONS AND METHODS FOR PREDICTING INTERFERON TREATMENT EFFICACY IN A SUBJECT

(71) Applicant: Yissum Research Development Company of the Hebrew University of Jerusalem Ltd., Jerusalem (IL)

(72) Inventor: Yoav SMITH, Jerusalem (IL)

(21) Appl. No.: 15/303,412

(22) PCT Filed: Apr. 2, 2015
(86) PCT No.: PCT/IL2015/050366

§ 371 Date: Oct. 11, 2016

Related U.S. Application Data
(60) Provisional application No. 61/977,977, filed on Apr. 10, 2014.

(51) Int. Cl. C12Q 1/68; G01N 33/68; G01N 33/50; C12Q 1/70

(52) U.S. Cl. CPC C12Q 1/6883; C12Q 1/706; G01N 33/6893; G01N 33/5023; C12Q 2600/106; C12Q 2600/118; C12Q 2600/158; G01N 2333/186; G01N 2800/52

(57) ABSTRACT

The present invention relates to kits, arrays, compositions and methods for predicting, assessing and evaluating responsiveness and success of interferon treatment as well as for monitoring disease progression and pathophysiology in a subject treated with interferon, using OAS2, HERC5, UPS18, UBE2L6 and optionally of ISG15 genes as biomarkers.

### TECHNOLOGICAL FIELD

The invention relates to personalized medicine. More specifically, the invention relates to kits, arrays, compositions and methods for predicting, assessing and evaluating responsiveness and success of interferon treatment of patients, specifically, patients infected with HCV.

### BACKGROUND REFERENCES

References considered to be relevant as background to the presently disclosed subject matter are listed below:
Chen Limin, et al., Gastroenterology 128:1437-1444 (2005).
Taylor, M W, et al., Journal of Virology 81:3391-3401 (2007).
Van Baarsen L G, et al., PLoS ONE 3:e1927 (2008).
Zeremski M, et al., J. Acquir. Immune. Defic. Syndr. 45:262-268 (2007).
Tarantino G, et al., Digestive and Liver Disease 40:A1-A40 (2008).
US2009/157324
WO10/076788
U.S. Pat. No. 6,258,569
U.S. Pat. No. 6,030,787
U.S. Pat. No. 5,952,202
U.S. Pat. No. 5,876,930
U.S. Pat. No. 5,866,336
U.S. Pat. No. 5,736,333
U.S. Pat. No. 5,723,591
U.S. Pat. No. 5,691,146
U.S. Pat. No. 5,538,848
Harlow and Lane, Antibodies: A Laboratory Manual, Cold Spring Harbor Laboratory, New York, 1988.

Acknowledgement of the above references herein is not to be inferred as meaning that these are in any way relevant to the patentability of the presently disclosed subject matter.

### BACKGROUND OF THE INVENTION

Interferon therapy is widely used in the treatment of a variety of diseases including for example, multiple sclerosis (MS), hepatitis B, hepatitis C, inflammatory diseases and many cancers types. However, not all subjects treated with interferon equally respond to this therapy and moreover, responsive subjects experience relapse of the disease after remission periods. In fact, in type 1 hepatitis C Virus (HCV) the success of treatment is only about 50%, namely about half of the patients administered with interferon will not benefit but rather experience only related side effects.

Evaluating the differences in the genetic profile of the two groups of patients can provides valuable insight in the interferon resistant mechanism.

Chen et al. 2005, compared the gene expression levels in liver specimens taken before treatment from 15 non-responders and 16 responders to Pegylated interferon (IFN-alpha), identified 18 genes that have a significantly different expression between all responders and all non-responders and concluded that up-regulation of a specific set of interferon-responsive gens predict non response to exogenous treatment.

Taylor M., et al. 2007, found that the induced levels of known interferon-stimulated genes such as the OAS1, OAS2, MX1, IRF-7 and TLR-7 genes is lower in poor-response patients than in marked- or intermediate-response patients.

Van Baarsen et al., 2008 show that the expression level of interferon response genes in the peripheral blood of multiple sclerosis patients prior to treatment can serve a role as a biomarker for the differential clinical response to interferon beta.

Zeremaki M., et al., 2007 showed that PEG-interferon induced elevations in IP-10 are greater in responders than in non-responders after the first PEG-interferon dose.

Tarantino et al., 2008 described that serum levels of B-Lymphocyes stimulator (BLyS) have a potential role as a predictor of outcome in patients with acute hepatitis C.

The Inventor's previous US Patent Application, US2009157324 describes a computational method for selecting a group of genes from a predetermined group of genes whose expression level is significantly different among a first group of individuals (being for example responders to a treatment) and comparing their expression in a second group of individuals (for example not responders). The statistical significance of each group of genes is determined in both up regulated genes or down regulated genes, namely their expression in the first group is higher or lower than in the second group, respectively. The genes in both groups (up regulated and down regulated) are ranked according to number of times each gene was ranked in the highest statistical significant score. A subset of genes having the highest score, either up regulated or down regulated are then selected as biomarkers.

In another Application by the Inventor, International Patent Publication WO10076788, computational and experimental methods are provided for predicting the responsiveness of a subject to interferon therapy by measuring the expression level of various genes such as OAS3, IF16, ISG15, OAS2, IFIT1, KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IFI27. Specifically, the inventor has found that OAS3, IF16, ISG15, OAS2 and IFIT1 are up-regulated in patients that do not respond to interferon treatment as compared to patients that respond to interferon therapy or compared to healthy controls.

New suitable biomarkers need to be considered for predicting response to therapy, predicting treatment success and monitoring disease prognosis and pathogenesis, specifically chances for disease relapse.

### SUMMARY OF THE INVENTION

According to a first aspect, the invention relates to a kit comprising detecting molecules specific for determining the level of expression of OAS2, HERC5, UPS18, UBE2L6 and optionally of ISG15, genes in a biological sample.

A second aspect of the invention relates to an array of detecting molecules specific for OAS2, HERC5, UPS18, UBE2L6 and optionally for ISG15, genes. In certain embodiments, the detecting molecules may be isolated detecting nucleic acid molecules and/or isolated detecting amino acid molecules.

According to a third aspect, the invention provides a prognostic composition comprising detecting molecules specific for determining the level of expression of OAS2, HERC5, UPS18, UBE2L6 and optionally of ISG15, genes in a biological sample.

In a further aspect, the invention relates to a prognostic method for predicting and assessing responsiveness of a mammalian subject to interferon treatment. The method comprising the steps of: (a) determining the level of expression of OAS2, HERC5, UPS18, UBE2L6 and optionally, of ISG15 genes in a biological sample of said subject to obtain an expression value. In the next step (b), determining if the expression value obtained in step (a), is positive or negative with respect to a predetermined standard expression value or to an expression value of said OAS2, HERC5, UPS18, UBE2L6 and optionally of ISG15, genes in at least one control sample.

It should be noted that a positive expression value of said OAS2, HERC5, UPS18, UBE2L6 and optionally of ISG15, genes as compared to the predetermined standard expression value or to the expression value of said genes in at least one control sample, indicates that the examined subject is not responsive to interferon treatment, thereby predicting responsiveness of a mammalian subject to interferon treatment.

These and other aspects of the invention will become apparent by the hand of the following drawings.

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIGS. 1A to 1F. are bar graphs showing the expression of UBE2L6 (FIG. 1A), USP18 (FIG. 1B), HERC5 (FIG. 1C), OAS2 (FIG. 1D), ISG15 (FIG. 1E) genes measured in PBMCs of HCV patients before initiation of treatment (day “0”). Expression data were obtained from RT-PCR measurements. FIG. 1F corresponds to the sum of the measured and normalized (GAPDH) expression of the five genes. The “X”-axis represents the subject number, wherein p6 and p7 correspond to HCV patients that show no response to treatment and p1, p2, p3, p4, p5 and p8 correspond to HCV patients that show response to treatment. The “Y” axis represents the normalized expression level of the genes.

FIG. 2. is a bar graph sum showing the normalized and scaled expression of the five genes UBE2L6, USP18, HERC5, OAS2 and ISG15 in each one of the tested patients and the amount of reduction in virus load.

FIGS. 3A to 3E. are bar graphs showing the normalized and scaled expression of OAS2 (FIG. 3A), HERC5 (FIG. 3B), UBE2L6 (FIG. 3C) and USP18 (FIG. 3D) genes measured in PBMCs of HCV patients before initiation of treatment (day “0”). Expression data were obtained from RT-PCR measurements. FIG. 3E corresponds to the sum of the normalized and scaled expression of the genes. The “X”-axis represents the subject number, wherein p6, p7 P212, P210 and P213 correspond to HCV patients that show no response to treatment and p1, p2, p3, p4, p5 and p8 correspond to HCV patients that show response to treatment. The “Y” axis represents the normalized and scaled expression level of the genes.

FIGS. 4A to 4I. are bar graphs showing the normalized and scaled expression of OAS2, HERC5, USP18, UBE2L6, ISG15, IFI27, IFI44L, UBE1L and IFIH1 genes measured in liver biopsies of HCV patients before initiation of treatment (day “0”). Expression data were obtained from RT-PCR measurements. The “X”-axis represents the subject number, and discriminate between an HCV patient that is predicted to show no response to treatment and HCV patient that is predicted to show response to treatment. The “Y” axis represents the normalized and scaled expression level of the genes.

FIG. 5. is a bar graph showing the sum of the normalized and scaled expression of the five genes OAS2, HERC5, USP18, UBE2L6, ISG15. The “X”-axis represents the subject number, distinguishing between HCV patients that show no response to treatment and HCV patients that show response to treatment. The “Y” axis represents the sum of the normalized and scaled expression level of the genes.

FIG. 6. is a bar graph showing the p-value obtained by t-test analysis of different sums of genes. The “X”-axis represents the tested group. The “Y” axis represents the p-value of each group.

FIG. 7. is a bar graph showing the p-value obtained by t-test analysis of the 4 ubiquitin genes plus a fifth different gene. The “X”-axis represents the tested group. The “Y” axis represents the p-value of each group.

FIG. 8. is a bar graph showing the p-value obtained by t-test analysis of sums of different selection of five genes. The “X”-axis represents the tested group. The “Y” axis represents the p-value of each group.

FIG. 9. is a ROC curve representing the response to interferon treatment in HCV patients combining the expression of five genes (OAS2, HERC5, USP18, UBE2L6, ISG15) measured before initiation of treatment (day “0”). ROC curves were plotted using Expression data obtained by RT-PCR

FIG. 10. is a partest graphical representations of the ROC results representing the response to interferon treatment in HCV patients using data from five genes (OAS2, HERC5, USP18, UBE2L6, ISG15) measured before initiation of treatment (day “0”). The columns represent false negative (bottom left column), true positive (top left column), false positive (top right column), true negative (bottom right column).

### DETAILED DESCRIPTION OF THE INVENTION

Predicting the chances of a patient to respond to treatment before initiation of treatment or at early stages after initiation of treatment is highly valuable and is desired clinically. The importance of adjusting suitable treatment protocols is appreciated in view of the fact that a large number of treatment protocols are often associated with some extent of undesired side effects. Thus, predicting response of a patient to a treatment protocol before and/or at early stages after initiation of treatment and/or throughout or after a treatment period may avoid inadequate treatments and reduce unnecessary side effects.

In addition, even if a patient responds to a specific treatment and experiences a remission period, it is not necessarily that the disease will not relapse at some later stages. Thus, identifying breakthrough points throughout the disease and even after remission can asses in predicting the probability of a disease relapse, which has proved to be one of the key for successful treatment of patients.

Interferon is widely clinically used for treatment of a variety of diseases including for example autoimmune diseases such as infectious diseases, for example, hepatitis C infection, immune-related disorders such as multiple sclerosis, different types of proliferative disorders and other inflammatory diseases. Significant therapeutic advances were made in the treatment of interferon associated diseases however, it is still difficult to determine at the time of disease diagnosis and treatment adjustments, which patients will respond to treatment and which would eventually relapse. Surprisingly, although interferon is considered as a state of art therapy in treatment of these diseases, many of the treated patients do not respond to the therapy and even if they do, many of the patients experience a relapse of the disease.

Thus, there is a critical need for reliable predictors that will provide gaudiness and identification of treatment success and failure, breakthrough point and predict inadequate treatments. In addition, responsiveness predictions provided throughout or after treatment periods enable development of alternatives dosing regimens of interferon.

In the present invention, the inventor has used computational tools and identified an arsenal of genes that is differently expressed in patients that were found to respond to interferon treatment and in patients that were found non-responders. In addition, this group of genes was also found to be differently expressed at different stages of disease, namely during infection and after or during treatment.

Specifically, as shown in Example 1 herein, the inventor has found that low expression of OAS2, HERC5, UPS18, UBE2L6 and optionally of ISG15 genes in blood samples obtained before treatment of HCV patients with interferon, is correlated with responsiveness to interferon, as reflected by reduction in virus load of the examined patients. In a similar manner, as shown in Example 2, low expression of OAS2, HERC5, UPS18, UBE2L6 and optionally of ISG15 genes in liver tissue samples obtained before treatment of HCV patients with interferon, is correlated with responsiveness to interferon.

The inventors have therefore concluded that the identified genes described herein are suitable for predicting, assessing and monitoring response of a patient, specifically, patient infected with HCV, to interferon treatment.

Thus, according to a first aspect, the invention provides a kit comprising: detecting molecules specific for determining the level of expression of OAS2, HERC5, UPS18, UBE2L6 and optionally of ISG15, genes in a biological sample.

Thus, in certain embodiments, the kit of the invention may comprise detecting molecules specific for OAS2, HERC5, UPS18 and UBE2L6 genes. In yet some other embodiments, the kit of the invention may comprise detecting molecules specific for OAS2, HERC5, UPS18, UBE2L6 and ISG15 genes.

According to some embodiments, the kit of the invention may further comprise at least one of: (a) detecting molecules specific for determining the level of expression of at least one control reference gene in a biological sample; (b) pre-determined calibration curve providing standard expression values of OAS2, HERC5, UPS18, UBE2L6 and optionally of ISG15, genes; (c) pre-determined calibration curve providing standard expression values of said at least one control reference gene; and (d) at least one control sample.

It should be noted that in certain embodiments, the control sample may be either a “negative” or a “positive” control. A “negative” or “positive” control is dependent upon the use of the kit. In certain embodiments “positive” control samples may be samples of known responsive subject/s, and “negative control samples may be samples of known non-responsive subject/s.

According to another embodiment, the kit of the invention may be a prognostic kit for predicting, assessing and monitoring responsiveness of a mammalian subject to interferon treatment. According to some embodiments, the subject is suffering from an infectious disease.

Still further, in certain embodiments, the infectious disease may be any one of viral diseases, protozoan diseases, bacterial diseases, parasitic diseases, fungal diseases and mycoplasma diseases. In a specific embodiment, the infectious disease is viral disease infection.

In certain embodiments, the kit of the invention is applicable for predicting and assessing responsiveness of a mammalian subject to interferon treatment of a subject suffering from an infectious disease caused by any one of hepatitis C, A or B virus (HCV, HAV, HBV), HIV, influenza (specifically, H1N1 and H5N1), dengue virus, West Nile virus (WNV), Polio virus. In specific embodiments, the kit of the invention may be particularly useful for predicting the responsiveness of subjects suffering from a hepatitis C virus (HCV) infection.

It should be appreciated that according to specific embodiments, the kit of the invention may be useful in predicting responsiveness to interferon treatment of subjects suffering from an autoimmune disease or a proliferative disorder.

According to certain embodiments, the autoimmune disease may be multiple sclerosis.

According to another embodiment, the kit of the invention may be applicable in cases that the tested subject is suffering from a proliferative disorder, for example, any one of melanoma, carcinoma sarcoma, glioma, leukemia and lymphoma. More specific embodiments relate to melanoma.

According to another embodiment, the kit of the invention may further comprise instructions for use. In more specific embodiments, such instructions may include at least one of: (a) instructions for carrying out the detection and quantification of expression of said OAS2, HERC5, UPS18, UBE2L6 and optionally of ISG15, genes; (b) instructions for carrying out the detection and quantification of expression of said at least one control reference gene; and (c) instructions for determining if the expression values of said OAS2, HERC5, UPS18, UBE2L6 and optionally of ISG15, genes is positive or negative with respect to a corresponding predetermined standard expression value of said genes.

In yet other specific embodiments the kit of the invention may comprise detecting molecules specific for the biomarkers of the invention, specifically, the OAS2, HERC5, UPS18, UBE2L6 and optionally of ISG15, genes. It should be further appreciated that the kit of the invention may further comprise detecting molecules specific for determining the level of expression of at least one of: IFI127, RSAD2, STAT1, EIF2AK2, IFI44L and UBEL1 genes.

According to another embodiment the detecting molecules comprised in the kit of the invention may be isolated detecting nucleic acid molecules, isolated detecting amino acid molecules or any combinations thereof.

In more specific embodiments, the kit of the invention may comprise nucleic acid based detecting molecules, specifically, isolated oligonucleotides, each oligonucleotide specifically hybridize to a nucleic acid sequence of a product, specifically, an RNA product of one of said OAS2, HERC5, UPS18, UBE2L6 and optionally of ISG15, genes. In an optional embodiment, the kit of the invention may further comprise nucleic acid based detecting molecules specific for a control reference gene. Such control gene may be used for normalizing the expression value measured in a specific test sample.

In yet other specific embodiments, the detecting molecules comprised in the kit of the invention may be at least one of at least one primer, at least one pair of primers, at least one nucleotide probe/s and any combinations thereof.

In optional embodiments, the kit of the invention may further comprise at least one reagent for conducting a nucleic acid amplification based assay. In certain embodiments, such assay may be selected from the group consisting of a Real-Time PCR (RT-PCR), micro arrays, PCR, in situ Hybridization and Comparative Genomic Hybridization.

In certain embodiments the kit of the invention may further comprise a solid support, wherein each of said detecting molecules is disposed in an array.

In more specific embodiments, the array of detecting molecules of the kit of the invention may comprise a plurality of addressed vessels.

In other alternative embodiments, the array of detecting molecules of the kit of the invention may comprise a solid support holding the detecting molecules in distinct regions. In certain embodiments, the distinct regions in said array are predetermined distinct regions.

It should be appreciated that the kit of the invention is suitable for determining the expression level of OAS2, HERC5, UPS18, UBE2L6 and optionally of ISG15, genes in a biological sample. In some embodiments the biological sample may be any one of a blood cells, blood, bone marrow, lymph fluid, serum, plasma, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents.

According to specific embodiments, the biological sample may be a blood sample. Specifically, the biological sample is a sample of peripheral blood mononuclear cells (PBMCs), or any subset of blood cells, for example, CD14⁺ cells.

It should be appreciated, that in case of blood sample, according to some embodiments, the kit of the invention may comprise detecting molecules specific for determining the level of expression of OAS2, HERC5, UPS18, UBE2L6 and optionally of ISG15, genes in said blood sample.

In yet other embodiments, in case the sample is a liver tissue biopsy sample, the kit may comprise detecting molecules specific for determining the level of expression of OAS2, HERC5, UPS18, UBE2L6 and ISG15 genes in said liver tissue biopsy sample.

The kit of the invention may therefore optionally comprise suitable means for obtaining said sample. More specifically, for using the kit of the invention, one must first obtain samples from the tested subjects, to do so, means for obtaining such samples may be required. Such means for obtaining a sample from the mammalian subject can be any means for obtaining a sample from the subject known in the art. Examples for obtaining e.g. blood or bone marrow samples are known in the art and could be any kind of finger or skin prick or lancet based device, which basically pierces the skin and results in a drop of blood being released from the skin. In addition, aspirating or biopsy needles may be also used for obtaining spleen lymph nodes tissue samples. Samples may of course be taken from any other living tissue, or body secretions comprising viable cells, such as biopsies, saliva or even urine.

It should be appreciated that the kit of the invention may be applicable for assessing and monitoring responsiveness of a subject suffering from a condition to a treatment with interferon. In such case, more than one sample should be obtained from different time points prior and after treatment. As will be discussed herein after the samples are referred to herein as “temporally separated samples”. The kit may further comprise as a further element, instructions for calculating the rate of change of the expression values (preferably, normalized values) of said marker genes of the invention, specifically, the OAS2, HERC5, UPS18, UBE2L6 and optionally of ISG15, genes, between said temporally-separated test samples. It should be noted that a positive rate of change of said expression values in a sample obtained after initiation of said treatment as compared to the marker genes expression value in a sample obtained prior to initiation of said treatment, is indicative of the responsiveness of said subject to said treatment.

The invention therefore provides a kit that is also applicable for a dynamic situation and is thus applicable for monitoring responsiveness and may be also used in monitoring the treated patients.

The inventors consider the kit of the invention in compartmental form. It should be therefore noted that the detecting molecules used for detecting the expression levels of OAS2, HERC5, UPS18, UBE2L6 and optionally of ISG15, genes may be provided in a kit attached to an array. As defined herein, a “detecting molecule array” refers to a plurality of detection molecules that may be nucleic acids based or protein based detecting molecules (specifically, probes, primers, antibodies or any combinations thereof), optionally attached to a support where each of the detecting molecules is attached to a support in a unique pre-selected and defined region.

For example, an array may contain different detecting molecules, such as specific antibodies, primers, and probes or any combinations thereof. As indicated herein after, in case a combined detection of the marker genes expression level is required and determined, the different detecting molecules for each target may be spatially arranged in a predetermined and separated location in an array. For example, an array may be a plurality of vessels (test tubes), plates, micro-wells in a micro-plate, each containing different detecting molecules, specifically, probes, primers, antibodies or any combinations thereof, specific against polynucleotide sequences or polypeptides encoded by the OAS2, HERC5, UPS18, UBE2L6 and optionally of ISG15, genes of the invention. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ Or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above, provided in the kit/s of the invention.

In one embodiment, the polynucleotide-based detection molecules of the invention may be in the form of nucleic acid probes, primers or any combinations thereof, which can be spotted onto an array to measure RNA from the sample of a subject to be diagnosed.

Thus, a second aspect of the invention relates to an array of detecting molecules specific for OAS2, HERC5, UPS18, UBE2L6 and optionally for ISG15, genes. It should be noted that the detecting molecules of the invention may be isolated detecting nucleic acid molecules or isolated detecting amino acid molecules, or any combination of both.

In certain specific embodiments, the array provided by the invention may be a nucleic acid array. As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules. In one embodiment, the nucleic acid member attached to the surface of the support is DNA. In a preferred embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, a “nucleic acid array” refers to a plurality of unique nucleic acid detecting molecules attached to nitrocellulose or other membranes used in Southern and/or Northern blotting techniques. For oligonucleotide-based arrays, the selection of oligonucleotides corresponding to the gene of interest which are useful as probes is well understood in the art.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “stably associated” or “stably bound” refers to a nucleic acid that is stably bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

As used herein, “substrate” or “support” or “solid support”, when referring to an array, refers to a material having a rigid or semi-rigid surface. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly) vinylidendifmoride, polystyrene, polycarbonate, a charged membrane, such as nylon or nitrocellulose, or combinations thereof. Preferably, at least one surface of the substrate may be substantially flat. The support may optionally contain reactive groups, including, but not limited to, carboxyl, amino, hydroxyl, thiol, and the like. In one embodiment, the support may be optically transparent. As noted above, the solid support may include polymers, such as polystyrene, agarose, sepharose, cellulose, glass, glass beads and magnetizable particles of cellulose or other polymers. The solid-support can be in the form of large or small beads, chips or particles, tubes, plates, or other forms.

According to some specific embodiments, the array of the invention may comprise a plurality of addressed vessels containing the detecting molecules.

In yet other embodiments, the array of the invention may comprise a solid support holding detecting molecules in distinct regions.

According to certain embodiments, the level of expression of the biomarkers of the invention, the OAS2, HERC5, UPS18, UBE2L6 and optionally of ISG15, genes, may be determined using a nucleic acid amplification assay. In some embodiments, such assay may be selected from the group consisting of: a Real-Time PCR, micro arrays, PCR, in situ Hybridization and Comparative Genomic Hybridization. It should be noted that the nucleic acid based procedures described herein after for the prognostic methods of the invention may be applicable also for any of the aspects of the invention.

In yet other alternative embodiments, the kits, arrays and compositions of the invention may comprise detecting amino acid molecules such as isolated antibodies, each antibody binds selectively to a protein product of said biomarkers of the invention, specifically, the OAS2, HERC5, UPS18, UBE2L6 and optionally of ISG15, genes. In such embodiments, the level of expression of said marker genes may be determined using an immunoassay selected from the group consisting of an ELISA, a RIA, a slot blot, a dot blot, immunohistochemical assay, FACS, a radio-imaging assay and a Western blot.

A third aspect of the invention relates to a prognostic composition comprising detecting molecules specific for determining the level of expression of OAS2, HERC5, UPS18, UBE2L6 and optionally of ISG15, genes in a biological sample. According to one embodiment, the prognostic composition of the invention is particularly useful for predicting, and assessing responsiveness of a mammalian subject to interferon treatment.

In an optional embodiment, the detecting molecules of said array may be attached to a solid support.

In certain embodiments, the prognostic composition of the invention comprises detecting molecules that are selected from isolated detecting nucleic acid molecules and isolated detecting amino acid molecules.

In other embodiments the detecting molecules may comprise isolated oligonucleotide/s, each oligonucleotide specifically hybridizes to a nucleic acid sequence of a product, specifically, an RNA product of one of said OAS2, HERC5, UPS18, UBE2L6 and optionally of ISG15, genes or to the product of one of said optional control reference genes.

More specifically, the detecting molecules may be at least one of at least one primer, at least one pair of primers, at least one nucleotide probe/s and any combinations thereof, as described herein after.

In certain embodiments, the compositions of the invention may further comprise detecting molecules specific for control reference gene. Such control reference gene may be used for normalizing the detected expression levels for OAS2, HERC5, UPS18, UBE2L6 and optionally of ISG15, genes of the invention.

Thus, according to a further aspect, the invention relates to a prognostic method for predicting, assessing and optionally monitoring responsiveness of a mammalian subject to interferon treatment.

In certain embodiments, the method of the invention comprises the steps of:

First, step (a) involves determining the level of expression of OAS2, HERC5, UPS18, UBE2L6 and optionally of ISG15, genes in a biological sample of said subject to obtain an expression value, or a sum of the expression values of the marker genes. The second step (b) involves determining if the expression value obtained in step (a), or the sum of these values, is positive or negative with respect to a predetermined standard expression value, or cutoff value.

Alternatively, the expression value of the sample may be compared to an expression value of said OAS2, HERC5, UPS18, UBE2L6 and optionally of ISG15, genes in at least one control sample.

Such control sample may be a sample obtained from at least one of a healthy subject, a subject suffering from an infection with other virus/s, a subject that responds to interferon treatment, or a non-responder HCV infected subject. The method of the invention thereby enables predicting assessing and monitoring responsiveness of a mammalian subject, specifically HCV patient to interferon treatment. In yet further alternative specific embodiments, the second step (b) of the method of the invention involves calculating and determining if the expression value obtained in step (a) is any one of, positive, negative or equal to a predetermined standard expression value, or cutoff value.

It should be noted that in certain embodiments, the expression level of OAS2, HERC5, UPS18, UBE2L6 and optionally of ISG15, genes of the invention may be determined prior to interferon treatment, during treatment or after interferon treatment. In specific embodiments, the method of the invention provides a “static” analysis, where only one sample obtained in only one time-point, preferably, prior to treatment (or alternatively, after a long period has been passed from the last treatment), is being examined.

In other specific embodiments, the method of the invention provides a “dynamic” analysis, where more than one sample is obtained in more than one time-point, preferably, at least one sample prior to treatment (or alternatively, after a long period has been passed from the last treatment), is being examined and at least one other sample obtained after the treatment is initiated.

The prognostic method of the invention is based on measuring and determining the expression level of OAS2, HERC5, UPS18, UBE2L6 and optionally of ISG15, genes, in a biological sample.

The terms “level of expression” or “expression level” are used interchangeably and generally refer to a numerical representation of the amount (quantity) of a polynucleotide or an amino acid product of said marker genes of the invention, specifically, OAS2, HERC5, UPS18, UBE2L6 and optionally of ISG15, genes, in a biological sample.

“Expression” generally refers to the process by which gene-encoded information is converted into the structures present and operating in the cell. For example, RNA (cDNA) expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of mRNA. Therefore, according to the invention “expression” of a gene, specifically, a gene encoding OAS2, HERC5, UPS18, UBE2L6 and optionally of ISG15, may refer to transcription into a polynucleotide or translation into polypeptide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

In certain and specific embodiments, the method of the invention further comprises an additional and optional step of normalization. According to this embodiment, in addition to determination of the level of expression of the biomarkers of the invention, specifically, the OAS2, HERC5, UPS18, UBE2L6 and optionally of ISG15, genes, the level of expression of at least one suitable control reference gene (e.g., housekeeping genes, such as GAPDH) is being determined in the same sample. According to such embodiment, the expression level of the biomarkers of the invention (OAS2, HERC5, UPS18, UBE2L6 and optionally of ISG15, genes) obtained in step (a) is normalized according to the expression level of said at least one reference control gene obtained in the additional optional step in said test sample, thereby obtaining a normalized expression value. Optionally, similar normalization is performed also in at least one control sample or a representing standard when applicable. The next step involves comparing the normalized expression value of said OAS2, HERC5, UPS18, UBE2L6 and optionally of ISG15, genes in the test biological sample obtained in this additional step, with a predetermined standard expression value, or a cut-off value, or with a normalized expression value of said marker genes in a control sample.

The term “expression value” refers to the result of a calculation, that uses as an input the “level of expression” or “expression level” obtained experimentally and by normalizing the “level of expression” or “expression level” by at least one normalization step as detailed herein, the calculated value termed herein “expression value” is obtained.

More specifically, as used herein, “normalized values” are the quotient of raw expression values of marker genes, namely, OAS2, HERC5, UPS18, UBE2L6 and optionally of ISG15, genes, divided by the expression value of a control reference gene from the same sample, such as a stably-expressed housekeeping control gene. Any assayed sample may contain more or less biological material than is intended, due to human error and equipment failures. Importantly, the same error or deviation applies to both the marker genes of the invention and to the control reference gene, for example, GAPDH or actin whose expression is essentially constant. Thus, division of the marker gene raw expression value (namely, OAS2, HERC5, UPS18, UBE2L6 and optionally of ISG15, genes) by the control reference gene raw expression value yields a quotient which is essentially free from any technical failures or inaccuracies (except for major errors which destroy the sample for testing purposes) and constitutes a normalized expression value of said marker gene. This normalized expression value may then be compared with normalized cutoff values, i.e., cutoff values calculated from normalized expression values. In certain embodiments, the control reference gene could be GAPDH, or any gene that maintains stable in all samples analyzed.

Normalized expression level values of the marker genes of the invention, namely, the OAS2, HERC5, UPS18, UBE2L6 and optionally of ISG15, genes (as well as a sum thereof), that are higher (positive) or lower (negative) in comparison with a corresponding predetermined standard expression value or a cut-off value in a control sample predict to which population of patients the tested sample belongs.

It should be appreciated that an important step in the prognostic method of the inventions is determining whether the normalized expression value of any one of the marker genes of the invention is changed compared to a pre determined cut off.

The second step of the method of the invention involves determining if the expression value obtained in step (a), is positive or negative with respect to a predetermined standard expression value or to an expression value of said OAS2, HERC5, UPS18, UBE2L6 and optionally of ISG15, genes or of a sum thereof, in at least one control sample. Such determination is performed by comparing the expression values determined for the tested sample with predetermined standard values or cutoff values, or alternatively, with expression values of said marker genes in a control sample. As used herein the term “comparing” denotes any examination of the expression level and/or expression values obtained in the samples of the invention as detailed throughout in order to discover similarities or differences between at least two different samples. It should be noted that comparing according to the present invention encompasses the possibility to use a computer based approach. In yet more specific embodiments, the second step (b) of the method of the invention involves calculating and determining if the expression value obtained in step (a) is any one of, positive, negative or equal to a predetermined standard expression value, or cutoff value. Such step involves calculating and measuring the difference between the expression values of the examined sample and the cutoff value and determining whether the examined sample can be defined as positive or negative.

As described hereinabove, the method of the invention refers to a predetermined cutoff value. It should be noted that a “cutoff value”, sometimes referred to simply as “cutoff” herein, is a value that meets the requirements for both high diagnostic sensitivity (true positive rate) and high diagnostic specificity (true negative rate).

It should be noted that the terms “sensitivity” and “specificity” are used herein with respect to the ability of one or more markers, specifically the OAS2, HERC5, UPS18, UBE2L6 and optionally of ISG15, genes, to correctly classify a sample as belonging to a pre-established population associated with responsiveness or alternatively, non-responsiveness to treatment or to a specific relapse rate.

“Sensitivity” indicates the performance of the bio-markers of the invention, the OAS2, HERC5, UPS18, UBE2L6 and optionally of ISG15, genes, with respect to correctly classifying samples as belonging to pre-established populations that are likely to respond to interferon therapy, wherein said bio-markers are consider here as OAS2, HERC5, UPS18, UBE2L6 and optionally of ISG15, genes.

“Specificity” indicates the performance of the bio-markers of the invention with respect to correctly classifying samples as belonging to pre-established populations that are likely to not respond to interferon treatment.

Simply put, “sensitivity” relates to the rate of correct identification of responsiveness in samples as such out of a group of samples, whereas “specificity” relates to the rate of correct identification of lack of responsiveness in samples as such out of a group of samples. Cutoff values may be used as a control sample, said cutoff values being the result of a statistical analysis of the marker genes expression values differences in pre-established populations healthy, responsive or nonresponsive.

Thus, a given population having specific clinical parameters will have a defined likelihood to respond to treatment based on the expression values of the marker genes, namely, the OAS2, HERC5, UPS18, UBE2L6 and optionally of ISG15, genes, being above or below said cutoff values. It should be emphasized that the nature of the invention is such that the accumulation of further patient data may improve the accuracy of the presently provided cutoff values, which are based on an ROC (Receiver Operating Characteristic) curve generated according to said patient data using, for example, the analytical software program developed by the inventor. The marker genes expression values are selected along the ROC curve for optimal combination of prognostic sensitivity and prognostic specificity which are as close to 100 percent as possible, and the resulting values are used as the cutoff values that distinguish between patients who will respond at a certain rate, and those who will not respond to the treatment (with said given sensitivity and specificity), to distinguish between responsive and non-responsive subjects. The ROC curve may evolve as more and more patient-responsiveness data and related gene expression values of said marker genes (specifically, OAS2, HERC5, UPS18, UBE2L6 and optionally of ISG15, genes) are recorded and taken into consideration, modifying the optimal cutoff values and improving sensitivity and specificity. Thus, the provided cutoff values should be viewed as a starting point that may shift as more responder and non-responder data allows more accurate cutoff value calculation. Although considered as initial cutoff values, the presently provided values already provide very good sensitivity and specificity, and are readily applicable in current clinical use, even in patients undergoing different treatment regimens.

As noted above, the expression value determined for the examined sample (or the normalized expression value) is compared with a predetermined cutoff or a control sample. More specifically, in certain embodiments, the expression value obtained for the examined sample is compared with a predetermined standard or cutoff value. In further embodiments, the predetermined standard expression value, or cutoff value has been pre-determined and calculated for a population comprising at least one of healthy subjects, subjects suffering from a disorder, specifically, HCV infected subjects that respond to interferon treatment, non-responder subjects, subjects in remission and subjects in relapse.

Still further, in certain alternative embodiments where a control sample is being used (instead of, or in addition to, pre-determined cutoff values), the normalized expression values of the marker genes used by the invention, specifically, OAS2, HERC5, UPS18, UBE2L6 and optionally of ISG15, genes, in the test sample are compared to the expression values in the control sample. In certain embodiments, such control sample may be obtained from at least one of a healthy subject, a subject suffering from an immune-related disorder, a subject that responds to interferon treatment and a non-responder subject.

Thus, in certain specific embodiments, the method of the invention may be specifically applicable for predicting responsiveness of a mammalian subject, specifically a subject infected with HCV, to interferon treatment. In such case, the method may comprise the steps of:

First (a), determining the level of expression of OAS2, HERC5, UPS18, UBE2L6 and optionally of ISG15, genes in at least one biological sample of the examined subject to obtain an expression value. In the second step (b), the expression value obtained in step (a) is compared with a predetermined standard expression value or cutoff value, thereby predicting responsiveness of a mammalian subject to interferon treatment. Alternatively, the expression value obtained for the examined sample may be compared with the expression value of the OAS2, HERC5, UPS18, UBE2L6 and optionally of ISG15, genes in at least one control sample, for example, a healthy, a responder and a non-responder subject. According to such embodiments, the level of expression of the marker genes of the invention is determined in at least one biological sample at any time before initiation of treatment and the obtained expression value is used to predict if the subject will respond to treatment. The expression value may be compared to an expression value of a population of subjects that respond to interferon treatment and/or to an expression value of a population of subjects that do not respond to interferon treatment. In yet further alternative specific embodiments, the second step (b) of the method of the invention involves calculating and determining if the expression value obtained in step (a) is any one of, positive, negative or equal to a predetermined standard expression value, or cutoff value.

Thus, in certain embodiments, a positive expression value, or in other words, a higher expression value of the biomarkers of the invention OAS2, HERC5, UPS18, UBE2L6 and optionally of ISG15, genes, as compared to the predetermined standard expression value (cutoff value), indicates that said subject belongs to a pre-established population associated with lack of responsiveness to interferon treatment and therefore, the subject may be considered as a non-responsive subject.

Alternatively, where the expression value of the examined subject is compared with the expression value of a control sample, for example, a population of subjects that respond to interferon treatment, a positive or higher expression value of the sample, indicates that the examined subject is a non-responsive subject. When the control sample is a population of non-responder subjects, a positive or equal expression value, indicates that the examined subject belongs to a population of subjects that lack responsiveness.

In further embodiments, where the expression value of the marker genes of the invention or of a sum thereof is “negative” or lower than a control sample or a standard value of non-responder subject/s, it is indicated that the examined subject belongs to responsive population. Still further, in cases the expression value of the marker genes or of a sum thereof is equal or lower with respect to the expression value of a control responsive subject or to the standard expression value of responders, the examined subject should be classified as belonging to responsive population.

It should be noted that according to this specific embodiment, for predicting responsiveness, determination of an expression value is performed prior to initiation of interferon treatment. It should be appreciated that in certain embodiments, samples may be also obtained from patients that were treated long time ago with interferon (specifically, months or years before the sample is taken).

As used herein the term “predicting responsiveness” refers to determining the likelihood that the subject will respond to interferon treatment, namely the success or failure of interferon treatment.

The term “response” or “responsiveness” to interferon treatment refers to an improvement in at least one relevant clinical parameter as compared to an untreated subject diagnosed with the same pathology (e.g., the same type, stage, degree and/or classification of the pathology), or as compared to the clinical parameters of the same subject prior to interferon treatment. As shown in Example 1, one parameter for evaluating responsiveness in case of HCV infection (or infection of any other virus), may be measuring reduction of virus load. In certain embodiments, in case of responsiveness, reduction of virus load in response to treatments should be a reduction by 100 or more, as shown in the Examples.

The term “non responder” to interferon treatment refers to a patient not experiencing an improvement in at least one of the clinical parameter and is diagnosed with the same condition as an untreated subject diagnosed with the same pathology (e.g., the same type, stage, degree and/or classification of the pathology, virus load), or experiencing the clinical parameters of the same subject prior to interferon treatment.

As detailed above, the prediction obtained by the method of the invention made by comparing between the sample and the patient population may be dependent on the selection of population of patients to which the sample is compared to. A positive or higher expression value of the sample over a population of responders indicates that the examined subject is a non-responsive subject.

In accordance with some embodiments, a positive expression value (or higher expression) of the marker genes reflects a high expression of said genes and is therefore indicative of a specific probability of lack of responsiveness to interferon treatment, said probability being higher than the specific probability of responsiveness in patients where the corresponding initial expression value of the marker genes are negative.

To disambiguate, a positive expression value indicates a higher risk for non-responsiveness to interferon treatment than a negative expression value. More particularly, the lack of responsiveness to interferon treatment is at least 1 percent, at least percent 2, at least 3 percent, at least 3 percent, at least 4 percent, at least 5 percent, at least 6 percent, at least 7 percent, at least 8 percent, at least 9 percent, at least 10 percent, at least 11 percent, at least 12 percent, at least 13 percent, at least 14 percent, at least 15 percent, at least 16 percent, at least 17 percent, at least 18 percent, at least 19 percent, at least 20 percent, at least 21 percent, at least 22 percent, at least 23 percent, at least 24 percent, at least 25 percent, at least 26 percent, at least 27 percent, at least 28 percent, at least 29 percent, at least 30 percent, at least 31 percent, at least 32 percent, at least 33 percent, at least 34 percent, at least 35 percent, at least 36 percent, at least 37 percent, at least 38 percent, at least 39 percent, at least 40 percent, at least 41 percent, at least 42 percent, at least 43 percent, at least 44 percent, at least 45 percent, at least 46 percent, at least 47 percent, at least 48 percent, at least 49 percent, at least 50 percent, at least 51 percent, at least 52 percent, at least 53 percent, at least 54 percent, at least 55 percent, at least 56 percent, at least 57 percent, at least 58 percent, at least 59 percent, at least 60 percent, at least 70 percent, at least 80 percent, at least 90 percent or more higher than the lack of responsiveness of patient population treated with interferon associated with the corresponding negative expression value (that reflects lower initial levels of expression of the marker genes of the invention, specifically, the OAS2, HERC5, UPS18, UBE2L6 and optionally of ISG15, genes, or of any sum of the expression of said marker genes.

In some embodiments, the term “specific probability” refers to a probability of a patient to respond to interferon treatment based on OAS2, HERC5, UPS18, UBE2L6 and optionally of ISG15, genes expression pattern, wherein the probability is calculated according to the patient population analysis provided herein, but may be further fine-tuned as more patient clinical data is accumulated and the same statistical analysis may be reiterated using the augmented clinical population database.

In some embodiments, the method of the invention comprises the step of determining the expression level of OAS2, HERC5, UPS18 and UBE2L6. In other embodiments, the method of the invention involves determining the expression level of OAS2, HERC5, UPS18, UBE2L6 and ISG15 genes.

Examples 1 and 2 herein below provides an example for a predetermined cut-off values of the marker genes expression that may be helpful in differentiating responders and non-responders and thus enable to predict response to interferon treatment, prior to initiation of treatment. High expression values, or “positive” expression values compared to this predetermined cut-off value are indicative of lack of response to treatment, whereas low expression values, or “negative” expression value, compared to this predetermined cut-off value are indicative of responsiveness to treatment.

As detailed below, it should be appreciated that the cut off value is highly dependent on the size of the tested averaged group as well as the extent of homogeneity and/or heterogeneity of the tested patients. Thus, determination of the cut off value is considered a dynamic computational process that is being iteratively verified and corrected.

It should be noted that the invention may further provide a method for assessing responsiveness of a mammalian subject to interferon treatment or evaluating the efficacy of interferon treatment on a subject. This method is based on determining the expression value of the biomarkers of the invention before and after initiation of interferon treatment, and calculating the ratio of the expression as a result of the treatment. The method therefore comprises the step of:

First, in step (a), determining the level of expression of OAS2, HERC5, UPS18, UBE2L6 and optionally of ISG15, genes in a biological sample of the examined subject to obtain an expression value. It should be noted that the sample is obtained prior to initiation of said treatment. The second step (b) involves determining the level of expression of said marker genes in at least one other biological sample of said subject, to obtain an expression value in said sample. This at least one other sample is obtained after initiation of said treatment. In the next step (c), calculating the rate of change between the expression value obtained in step (a) before initiation, and the expression value obtained in step (b), after the initiation of the treatment. It should be noted that for determining the rate of change, the ratio between the expression value of a sample obtained after initiation of the treatment, and the expression value of a sample obtained before initiating interferon treatment, is calculated. In certain embodiments, the ratio may be calculated between the expression values of a sample obtained before to the expression value of a sample obtained after initiation of interferon treatment. In the next step (d), the rate of change obtained in step (c) is compared with a predetermined standard rate of change determined between at least one sample obtained prior to and at least one sample obtained following interferon treatment. As an alternative to the use of a predetermined cutoff value of such rate of change, the method of the invention may involve the use of at least one control sample, and the rate of change calculated for the examined subject will be compared to the rate of change calculated for expression values in at least one control sample obtained prior and following interferon treatment.

In yet a further specific embodiments, the fourth step (d) of the method of the invention involves calculating and determining if the rate of change obtained in step (c) is any one of, positive, negative or equal to a predetermined standard rate of change.

It should be noted that a positive rate of change in the expression values of said OAS2, HERC5, UPS18, UBE2L6 and optionally of ISG15, genes in said sample as compared to a predetermined standard rate of change (predetermined cutoff of the rate of change), or to the rate of change calculated for expression values in at least one control sample obtained prior and following interferon treatment, indicates that the examined subject belongs to a pre-established population associated with responsiveness to interferon treatment. Such result is therefore indicative of a successful therapy. This method thereby provides assessing responsiveness of a mammalian subject to interferon treatment or evaluating the efficacy of interferon treatment on a subject.

According to such embodiments, the method of the invention further provides a tool for selecting an interferon treatment regimen for treating a subject diagnosed with a condition, by assessing and evaluating the efficacy of interferon treatment given to a subject suffering from condition to be treated, and selecting an interferon treatment regimen based on the evaluation; thereby selecting the treatment regimen for treating the subject diagnosed with a condition.

As used herein the phrase “assessing the responsiveness or evaluating efficacy of interferon treatment” refers to determining the likelihood (predicting) that interferon treatment is efficient or non-efficient in treating a specific condition, e.g., the success or failure of the treatment in treating the condition in a subject in need thereof. The term “efficacy” as used herein refers to the extent to which interferon treatment produces a beneficial result, e.g., an improvement in one or more symptoms of the pathology (caused by the condition to be treated) and/or clinical parameters related to the pathology as described herein below. For example, the efficacy of interferon treatment may be evaluated using standard therapeutic indices for each condition separately being for example, an infectious disease (or alternatively, an autoimmune disease or a proliferative disorder).

According to some embodiments of the invention, the efficacy of interferon treatment is a long-term efficacy. As used herein the phrase “long-term efficacy” refers to the ability of a treatment to maintain a beneficial result over a period of time, e.g., at least about 16 weeks, at least about 26 weeks, at least about 32 weeks, at least about 36 weeks, at least about 40 weeks, at least about 48 weeks, at least about 52 weeks, at least about 18 months, at least about 24 months, at least about 3 years, at least about 4 years, at least about 5 years, at least about 6 years, at least about 7 years, at least about 8 years, at least about 9 years, at least about 10 years, or longer.

According to some embodiments of the invention, a treatment with interferon that either directly or indirectly affects the condition to be treated, is considered efficient in treating a condition if it exerts an improvement in at least one relevant clinical parameter related to said condition in the treated subject as compared to an untreated subject diagnosed with the same condition (e.g., where the condition is an infectious disease, such parameter may include reduction of virus load), or as compared to the clinical parameters related to the said condition of the same subject prior to the interferon treatment.

By obtaining at least two and preferably more biological samples from a subject and analyzing them according to the method of the invention, the prognostic method may be effective for assessing responsiveness to treatment by monitoring molecular alterations indicating a success or failure of treatment in said patient. Thus, the prognostic method of the invention may be applicable for early assessment. Prior as used herein is meant the first time point is at any time before initiation of treatment, ideally several minutes before initiation of treatment. However, it should be noted that any time point before initiation of the treatment, including hours, days, weeks, months or years, may be useful for this method and is therefore encompassed by the invention. The second time point is collected from the same patient after hours, days, weeks, months or even years after initiation of treatment. More specifically, at least 0.5 hour, at least 1 hour, at least two hours, at least 3 hours, at least 4 hours, at least 6 hours, at least 10 hours, at least 12 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 40 days, at least 50 days, at least 60 days, at least 70 days, at least 78 days, at least 80, at least 90 days, at least 100 days, at least 110, at least 120 days, at least 130 days, at least 140 days or at least 150 days after initiation of treatment.

In some embodiments, the second time point is obtained between 1 minute or immediately after treatment to 24 month after initiation of the treatment. In some other embodiments, the second time point is between 1 hour to 6 hours after initiation of the treatment. In yet some other embodiments, the second time point is between 1 month to 3 month after initiation of the treatment.

In practice, for assessing response to interferon treatment, at least two test samples (before and after treatment) must be collected from the treated patient, and preferably more. The expression level of OAS2, HERC5, UPS18, UBE2L6 and optionally of ISG15, genes is then determined using the method of the invention, applied for each sample. As detailed above, the expression value is obtained from the experimental expression level. The rate of change of each biomarker expression, namely, OAS2, HERC5, UPS18, UBE2L6 and optionally of ISG15, genes or a sum thereof, is then calculated and determined by dividing the two expression values obtained from the same patient in different time-points or time intervals one by the other.

It should be noted that it is possible to divide the prior-treatment expression value by the after treatment expression value and vise versa. For the sake of clarity, as used herein, the rate of change is referred as the ratio obtained when dividing the expression value obtained at the later time point of the time interval by the expression value obtained at the earlier time point (for example before initiation of treatment).

For example, this interval may be at least one day, at least three days, at least three days, at least one week, at least two weeks, at least three weeks, at least one month, at least two months, at least three months, at least four months, at least five months, at least one year, or even more. Permeably the second point is obtained at the earlier time point that can provide valuable information regarding assessing response of the patient to interferon treatment.

As detailed above, this rate of change calculated for the examined sample is compared with a predetermined standard rate of change. The predetermined standard rate of change may be determined between at least one sample obtained prior to and at least one sample obtained following interferon treatment. It must be recognized that these predetermined rates of change were calculated for populations described herein and therefore reflect the rate in said specific population. As an alternative to the use of a predetermined cutoff value of such rate of change, the method of the invention may involve the use of at least one control samples, and the rate of change calculated for the examined subject may be compared to the rate of change calculated for expression values in at least one control sample obtained prior and following interferon treatment.

In yet further alternative specific embodiments, the fourth step (d) of the method of the invention involves calculating and determining if the rate of change obtained in step (c) is any one of, positive, negative or equal to a predetermined standard rate of change.

In accordance with some embodiments, a positive rate of change of OAS2, HERC5, UPS18, UBE2L6 and optionally of ISG15, genes expression values as compared to the predetermined standard rate of change is indicative of a specific probability to respond to interferon treatment, said probability being higher than the specific probability of responsiveness in patients where the corresponding rate of change of OAS2, HERC5, UPS18, UBE2L6 and optionally of ISG15, genes expression values is negative.

Similarly, a negative or equal rate of change in the expression value of said marker genes as compared to a predetermined standard rate of change is indicative of a specific probability of non-responsiveness to interferon treatment, said probability being higher than the specific probability of non-responsiveness in patients where the corresponding rate of change of said OAS2, HERC5, UPS18, UBE2L6 and optionally of ISG15, genes is positive.

To disambiguate, a positive rate of change in the expression value of said OAS2, HERC5, UPS18, UBE2L6 and optionally of ISG15, genes indicates a higher probability for responsiveness to interferon treatment than an equal or negative rate of change in the expression value of said marker genes. More particularly, responsiveness to interferon treatment is at least 1 percent, at least percent 2, at least 3 percent, at least 3 percent, at least 4 percent, at least 5 percent, at least 6 percent, at least 7 percent, at least 8 percent, at least 9 percent, at least 10 percent, at least 11 percent, at least 12 percent, at least 13 percent, at least 14 percent, at least 15 percent, at least 16 percent, at least 17 percent, at least 18 percent, at least 19 percent, at least 20 percent, at least 21 percent, at least 22 percent, at least 23 percent, at least 24 percent, at least 25 percent, at least 26 percent, at least 27 percent, at least 28 percent, at least 29 percent, at least 30 percent, at least 31 percent, at least 32 percent, at least 33 percent, at least 34 percent, at least 35 percent, at least 36 percent, at least 37 percent, at least 38 percent, at least 39 percent, at least 40 percent, at least 41 percent, at least 42 percent, at least 43 percent, at least 44 percent, at least 45 percent, at least 46 percent, at least 47 percent, at least 48 percent, at least 49 percent, at least 50 percent, at least 51 percent, at least 52 percent, at least 53 percent, at least 54 percent, at least 55 percent, at least 56 percent, at least 57 percent, at least 58 percent, at least 59 percent, at least 60 percent, at least 70 percent, at least 80 percent, at least 90 percent or more higher than the lack of responsiveness of patient population treated with interferon associated with the corresponding positive rate of change in the expression value of said OAS2, HERC5, UPS18, UBE2L6 and optionally of ISG15, genes.

Accordingly, the present invention provides a highly accurate determination of responsiveness as early as at the time of diagnosis, before initiation of treatment, and in fact, may assist in determining the optimal treatment.

As appreciated, the predetermined rate of change calculated for a pre-established population as detailed above for example encompasses a range for the rate of change having a low value and a high value, as obtained from a population of individuals including healthy controls, responders and non-responders. Thus, a subgroup of responsive patients can be obtained from the entire tested population. In this pre-established responsive population, the low value may be characterized by a low response whereas the high value may be associated with a high response as indicated by regular clinical evaluation. Therefore, in addition to assessing responsiveness to treatment, the rate of change may provide insight into the degree or extent of responsiveness. For example, a calculated rate of change that is closer in its value to the low value may be indicative of a low response and thus although the patient is considered responsive, increasing dosing or frequency of administration may be considered. Alternatively, a calculated rate of change that is closer in its value to the high value may be indicative of a high response, even at times leading to remission and thus lowering the administration dosage may be considered.

For clarity, when referring to a pre-established population associated with responsiveness, it is meant that a statistically-meaningful group of patients treated with interferon was analyzed as disclosed herein, and the correlations between OAS2, HERC5, UPS18, UBE2L6 and optionally of ISG15, genes expression values (and optionally other patient clinical parameters) and responsiveness to interferon treatment was calculated. For example, a specific fraction of a group of patients, which was found to have a positive rate of change in the expression values of said marker genes over the cutoff values according to the invention, was found to be responsive. Thus, responsiveness is associated with a population characterized by initial low expression levels of said OAS2, HERC5, UPS18, UBE2L6 and optionally of ISG15, genes that are elevated in response to interferon treatment, said population is a pre-established population, that is, a defined population whose responsiveness is known. Moreover, the populations may be defined by the expression (or a sum the expression values) of the OAS2, HERC5, UPS18, UBE2L6 and optionally of ISG15, genes vis a vis the cutoff values determined by the invention. The population may optionally be further divided into sub-populations according to other patient parameters, for example gender and age.

The method of the invention may be used for personalized medicine, namely adjusting and customizing healthcare with decisions and practices being suitable to the individual patient by use of genetic information and any additional information collected at different stages of the disease.

In yet another alternative embodiment, for assessing responsiveness of a mammalian subject to interferon treatment or evaluating the efficacy of interferon treatment on a subject suffering from a pathologic condition, the method of the invention may comprise:

(a) determining the level of expression of the marker genes of the invention in a biological sample of said subject to obtain an expression value, wherein said sample is obtained prior to initiation of said treatment;
(b) determining the level of expression of said marker genes in at least one other biological sample of said subject, to obtain an expression value, wherein said at least one other sample is obtained after initiation of said treatment;
(c) comparing the expression value obtained in step (a), with the expression value obtained in step (b), or in yet further alternative specific embodiments, calculating and determining if the expression value obtained in step (a) is any one of, positive, negative or equal to the expression value obtained in step (b).

Wherein a higher expression value of OAS2, HERC5, UPS18, UBE2L6 and optionally of ISG15, genes (or of a sum thereof) in a sample obtained after initiation of said treatment according to step (b) as compared to the expression value in a sample obtained prior to initiation of said treatment according to step (a), indicates that said subject belongs to a pre-established population associated with responsiveness to interferon treatment.

In accordance with such an embodiment, a patient diagnosed with a disease in need for interferon treatment is examined and a sample is obtained before initiation of treatment, the patient is then treated with interferon according to common treatment protocol and at any time point after treatment an additional sample is obtained from the patient. The second sample may be obtained after at least 3 hours, at least 4 hours, at least 6 hours, at least 10 hours, at least 12 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 40 days, at least 50 days, at least 60 days, at least 70 days, at least 78 days, at least 80, at least 90 days, at least 100 days, at least 110, at least 120 days, at least 130 days, at least 140 days or at least 150 days after initiation of treatment.

The first sample may be analyzed at the time it was obtained from the patient or alternatively may be kept under appropriate conditions for example, under freezing conditions, or as a paraffin embedded sample. The two samples are equally analyzed, optionally at the same time, for determining the expression of the marker genes of the invention. The data obtained as an expression value are compared by normalization of the expression level as detailed herein.

Patient having a “positive” expression value (or sum of expression values of these genes) that is a higher expression value of said marker genes of the invention in a sample obtained after initiation of said treatment as compared to the expression value in a sample obtained prior to initiation of said treatment according to step (a) belong to a pre-established population associated with responsiveness to interferon treatment.

In yet other embodiments, the invention provides a method for monitoring disease progression or early prognosis for disease relapse. According to certain embodiments, said method comprises the steps of: First (a), determining the level of expression of OAS2, HERC5, UPS18, UBE2L6 and optionally of ISG15, genes in a biological sample of said subject to obtain an expression value. The next steps involve (b) repeating step (a) to obtain expression values of OAS2, HERC5, UPS18, UBE2L6 and optionally of ISG15, genes, for at least one more temporally-separated test sample. The rate of change of the expression values of said marker genes are then calculated in step (c) between said temporally-separated test samples.

In the next step (d), the rate of change obtained in step (c) is compared with a predetermined standard rate of change (cutoff value) determined for expression value between samples obtained from at least one subject in remission and in relapse following interferon treatment or to the rate of change calculated for expression values in at least one control sample obtained in remission and in relapse following interferon treatment. It should be appreciated that in an alternative embodiment, step (d) of the method of the invention involves calculating and determining if the rate of change obtained in step (c) is any one of, positive, negative or equal to a predetermined standard rate of change.

According to certain embodiments, a negative rate of change in the expression values of OAS2, HERC5, UPS18, UBE2L6 and optionally of ISG15, genes in said sample as compared to a predetermined standard rate (cutoff) of change or to the rate of change calculated for expression values in said at least one control sample, indicates that said subject belongs to a pre-established population associated with relapse, thereby indicating that the examined subject is in relapse.

Thus, according to such embodiments, the method of the invention further provides early prognosis/diagnosis for monitoring disease relapse.

The term “relapse”, as used herein, relates to the re-occurrence of a condition, disease or disorder that affected a person in the past. Specifically, the term relates to the re-occurrence of a disease being treated with interferon.

Prognosis is defined as a forecast of the future course of a disease or disorder, based on medical knowledge. This highlights the major advantage of the invention, namely, the ability to predict relapse rate in patients as soon as they are diagnosed, even prior to treatment, based on a specific genetic fingerprinting of a patient. This early prognosis facilitates the selection of appropriate treatment regimens that may minimize the predicted relapse, individually to each patient, as part of personalized medicine.

As indicated above, in accordance with some embodiments of the invention, in order to asses response to interferon treatment at least two “temporally-separated” test samples must be collected from the treated patient and compared thereafter in order to obtain the rate of expression change in the marker genes of the invention, specifically, the OAS2, HERC5, UPS18, UBE2L6 and optionally of ISG15, genes. In practice, to detect a change the marker genes expression, at least two “temporally-separated” test samples and preferably more must be collected from the patient.

The expression of at least one of the markers is then determined using the method of the invention, applied for each sample. As detailed above, the rate of change in marker expression is calculated by determining the ratio between the two expression values, obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the dieses.

The term “remission”, as used herein, relates to the state of absence of disease activity in patients known to have un-curable chronic illness. It is commonly used to refer to absence of active infectious disease when this disease is expected to manifest again in the future. A partial remission may be defined for cancer as 50 percent or greater reduction in the measurable parameters of the disease (for example, virus load) as may be found on physical examination, radiologic study, or by biomarker levels from a blood or urine test. A complete remission is defined as complete disappearance of all such manifestations of disease. Each disease or even clinical trial can have its own definition of a partial remission.

When calculating the rate of change, one may use any two samples collected at different time points from the patient. To ensure more reliable results and reduce statistical deviations to a minimum, averaging the calculated rates of several sample pairs is preferable. A calculated or average negative rate of change of the expression values of said marker genes of the invention (specifically, OAS2, HERC5, UPS18, UBE2L6 and optionally of ISG15, genes), indicates that the subject is in relapse. It should be noted that in certain embodiments, where normalization step is being performed, the expression values referred to above, are normalized expression values.

As indicated above, in order to execute the prognostic method of the invention, at least two different samples must be obtained from the subject in order to calculate the rate of change in the expression of the marker genes. By obtaining at least two and preferably more biological samples from a subject and analyzing them according to the method of the invention, the prognostic method may be effective for predicting, monitoring and early diagnosing molecular alterations indicating a relapse in said patient.

Thus, the prognostic method may be applicable for early, sub-symptomatic diagnosis of relapse when used for analysis of more than a single sample along the time-course of diagnosis, treatment and follow-up.

An “early diagnosis” provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

Simply put, a decline or no change in the expression of the OAS2, HERC5, UPS18, UBE2L6 and optionally of ISG15, genes of the invention, indicate a relapse, and may provide an early sign before over symptoms occur, allowing for a quicker and more efficient therapeutic response.

Of course, more samples taken in more time-points may provide a statistically robust analysis of said expression trends, and may also be utilized as a method for continuous monitoring of subjects, especially those still undergoing and those that have undergone therapy. The more samples are available over a given time period, the higher is the resolution of the expression patterns of said marker genes during said period.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in expression rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more. In one particular example, the samples are taken from the monitored subject every two months for a period of 5 years.

Nevertheless, the present invention shows that OAS2, HERC5, UPS18, UBE2L6 and optionally of ISG15, genes may serve as prognostic markers for responsiveness to interferon treatment, and optionally for predicting and monitoring relapse in patients treated with interferon. These markers were shown as independent markers that are not affected by clinical parameters or treatment regimen. The expression “associated with a specific relapse rate”, “linked to a specific relapse rate” or “associated with a relapse rate” or similar expressions refer to a statistical connection between the expression values the marker genes of the invention, namely, the OAS2, HERC5, UPS18, UBE2L6 and optionally of ISG15, genes, the clinical parameters and a specific relapse rate, or the patient population which is known to relapse in that rate.

The method for monitoring disease progression or early prognosis for disease relapse as detailed herein may be used for personalized medicine, by collecting at least two samples from the same patient at different stages of the disease.

Thus, in yet another alternative embodiment for monitoring disease progression or early prognosis of disease relapse on a subject suffering from a condition, the method of the invention may comprise:

(a) determining the level of expression of OAS2, HERC5, UPS18, UBE2L6 and optionally of ISG15, genes in a biological sample of said subject to obtain an expression value, wherein said sample is obtained at any time point after initiation of said treatment;
(b) determining the level of expression of said marker genes in at least one other biological sample of said subject, to obtain an expression value, wherein said at least one other sample is obtained at a different time point after initiation of said treatment;
(c) comparing the expression value obtained in step (a), with the expression value obtained in step (b); or calculating and determining if the expression value obtained in step (b) is any one of, positive, negative or equal to the expression value obtained in (a).

Wherein a lower (negative) expression value of said marker genes (or of a sum thereof) in a sample obtained at a later time point after initiation of the treatment according to step (b) as compared to the expression value in a sample obtained at an earlier time point after initiation of said treatment according to step (a), indicates that said subject may be considered in a relapse.

In any case, a reduction in the moralized expression values of the marker genes of the invention, OAS2, HERC5, UPS18, UBE2L6 and optionally of ISG15, genes, indicates a relapse, alternatively, an increase in the normalized expression values of said marker genes (or in a sum thereof) may indicate an improvement in the clinical condition of the subject, i.e., that the patient is in remission. When using the method described herein for personalized medicine, it is appreciated that the more samples obtained at different time point, the more reliable the prediction for relapse would be.

In certain specific embodiments, if no change (or at least a statistical change) is observed in the rate of change of the expression values of the biomarkers of the invention, OAS2, HERC5, UPS18, UBE2L6 and optionally of ISG15, genes, as compared to a respective predetermined standard rate of change, an additional sample from the same patient may be obtained at a later time point. Responsiveness, remission or relapse may be assessed based on the information obtained from the two measurements.

As shown by the following examples, the level of expression of the genes of the kit of the invention reflects the ability of a specific individual to respond to a certain treatment, specifically, interferon treatment. Therefore, it should be appreciated that the kit of the invention as well as the methods disclosed herein may further provide a tool for evaluating the extent of responsiveness of a specific individual to a specific treatment regimen. More specifically, an individual displaying lower level of expression may exhibit a more effective response to a certain treatment regimen, and therefore may require a reduced treatment regimen. In the same manner, a responsive individual showing higher levels of expression (although within the range of the responsive population), may exhibit a less effective response and thus may require an extended treatment regimen. As such, the kits and methods of the invention provide a clear identification of responsive individuals and also a tool for evaluating the extent of the predicted response in a given individual.

The kits, arrays, compositions and methods of the invention described herein, relate to interferon treatment, specifically, to assessing the responsiveness to interferon treatment. As used herein the term “interferon” or “IFN” which is interchangeably used herein, refers to a synthetic, recombinant or purified interferon, and encompasses interferon type I that binds to the cell surface receptor complex IFN-α receptor (IFNAR) consisting of IFNAR1 and IFNAR2 chains; interferon type II that binds to the IFNGR receptor; and interferon type III, that binds to a receptor complex consisting of IL10R2 (also called CRF2-4) and IFNLR1 (also called CRF2-12).

Interferon type I in human includes interferon alpha 1 (GenBank Accession No. NM_024013 and NP_076918; SEQ ID NOs: 1 and 2 respectively), interferon alpha 2 (GenBank Accession No. NM_000605 and NP_000596; SEQ ID NO: 3 and 4, respectively), Interferon alpha-4 (GenBank Accession No. NM_021068 and NP_066546; SEQ ID NO: 5 and 6, respectively), Interferon alpha-5 (GenBank Accession No. NM_002169 and NP_002160; SEQ ID NO: 7 and 8, respectively), Interferon alpha-6 (GenBank Accession No. NM_021002 and NP_066282; SEQ ID NO: 0 and 10, respectively), Interferon alpha-7 (GenBank Accession No. NM_021057 and NP_066401; SEQ ID NO: 11 and 12, respectively), Interferon alpha-8 (GenBank Accession No. NM_002170 and NP_002161; SEQ ID NO: 13 and 14, respectively), Interferon alpha-10 (GenBank Accession No. NM_002171 and NP_002162; SEQ ID NO: 15 and 16, respectively), Interferon alpha-1/13 (GenBank Accession No. NM_006900 and NP_008831; SEQ ID NO: 17 and 18, respectively), Interferon alpha-14 (GenBank Accession No. NM_002172 and NP_002163; SEQ ID NO: 19 and 20, respectively), Interferon alpha-16 (GenBank Accession No. NM_002173 and NP_002164; SEQ ID NO: 21 and 22, respectively), Interferon alpha-17 (GenBank Accession No. NM_021268 and NP_067091; SEQ ID NO: 23 and 24, respectively) and Interferon alpha-21 (GenBank Accession No. NM_002175 and NP_002166; SEQ ID NO: 25 and 26, respectively), Interferon, beta 1 (GenBank Accession No. NM_002176 and NP_002167; SEQ ID NO: 27 and 28, respectively), and Interferon omega-1 (GenBank Accession No. NM_002177 and NP_002168; SEQ ID NOs: 29 and 30 respectively)].
Interferon type II in humans is Interferon-gamma (GenBank Accession No. NM_000619 and NP_000610; SEQ ID NOs: 31 and 32 respectively).

As used herein the phrase “interferon treatment” refers to administration of interferon into a subject in need thereof. It should be noted that administration of interferon may comprise a single or multiple dosages, as well as a continuous administration, depending on the pathology to be treated and a clinical assessment of the subject receiving the treatment.

Various modes of interferon administration are known in the art. These include, but are not limited to, injection (e.g., using a subcutaneous, intramuscular, intravenous, or intradermal injection), intranasal administration and oral administration.

According to some embodiments of the invention, interferon treatment is provided to the subject in doses matching his weight, at a frequency of once a week, for a period of up to 48 weeks.

Non-limiting examples of interferon treatment and representative diseases includes the following interferon beta-1a, interferon beta-Ib, recombinant IFN-a2b.

As appreciated in the art, interferon alfa-2a treatment is known as Roferon. Interferon alpha 2b treatment is by Intron A or Reliferon or Uniferon. Interferon beta-1a is sold under the trade names Avonex and Rebif. CinnaGen is a biosimilar compound. Interferon beta-1b is sold under trade names Betaferon, Betaseron, Extavia and ZIFERON.

Interferon treatment may comprise PEGylated interferon i.e., conjugated to a polyethylene glycol (PEG) polymer. For example, PEGylated interferon alpha 2a is sold under the trade name Pegasys. PEGylated interferon alpha 2a in Egypt is sold under the trade name Reiferon Retard. PEGylated interferon alpha 2b is sold under the trade name PegIntron.

The interferon treatment can also comprise a combination of interferon and ribavirin. For example, PEGylated interferon alpha 2b plus ribavirin is sold under the trade name Pegetron.

The invention shows that the expression levels of the biomarkers of the invention, OAS2, HERC5, UPS18, UBE2L6 and optionally of ISG15, genes, may be used as a prognostic tool distinguishing between interferon responders and non-responders and between subjects in relapse and subjects in remission.

Still further, as shown by the Examples, a group of genes, namely, OAS2, HERC5, UPS18, UBE2L6 and optionally of ISG15, genes, were shown as discriminating between populations of responders and non-responders. However, in it should be appreciated that in certain embodiments, further markers may be added to these biomarker genes of the invention. Thus, in yet another embodiment, the marker genes of the invention may comprise in addition at least one of IFI127, RSAD2, STAT1, EIF2AK2, IFI44L and UBE1L genes.

More specifically, it must be appreciated that the method of the invention may determine and use as a prognostic tool the expression value of any of the marker genes of the invention, OAS2, HERC5, UPS18, UBE2L6 and optionally of ISG15, described herein below.

2′-5′-oligoadenylate synthetase 2 (OAS2) gene (GenBank Accession No. NM_016817 SEQ ID NO:33, NM_002535 SEQ ID NO:34, NM_001032731 SEQ ID NO:35) encodes the OAS2 protein (GenBank Accession No. NP_058197 SEQ ID NO:36, NP_002526 SEQ ID NO:37, NP_001027903 SEQ ID NO:38).

HECT and RLD domain containing E3 ubiquitin protein ligase 5 (HERC5) gene (GenBank Accession No. NM_016323; SEQ ID NO: 39) encodes the HERC5 protein (GenBank Accession No. NP_057407 SEQ ID NO: 40). HERC5 gene is a member of the HERC family of ubiquitin ligases and encodes a protein with a HECT domain and five RCC1 repeats. Pro-inflammatory cytokines up regulate expression of this gene in endothelial cells. The HERC5 protein localizes to the cytoplasm and perinuclear region and functions as an interferon-induced E3 protein ligase that mediates ISGylation of protein targets. It is a major E3 ligase for ISG15 conjugation. HERC5 Acts as a positive regulator of innate antiviral response in cells induced by interferon. Makes part of the ISGylation machinery that recognizes target proteins in a broad and relatively non-specific manner.

Ubiquitin specific peptidase 18 (USP18) gene (GenBank Accession No. MN_017414; SEQ ID NO: 41) encodes the USP18 protein (GenBank Accession No. NP_059110 SEQ ID NO: 42). The protein encoded by this gene belongs to the ubiquitin-specific proteases (UBP) family of enzymes that cleave ubiquitin from ubiquitinated protein substrates. It is highly expressed in liver and thymus, and is localized to the nucleus. USP18 protein efficiently cleaves only ISG15 (an ubiquitin-like protein) fusions, and deletion of this gene in mice results in a massive increase of ISG15 conjugates in tissues, indicating that this protein is a major ISG15-specific protease.

Mice lacking this gene are also hypersensitive to interferon, suggesting a function of this protein in down regulating interferon responses, independent of its isopeptidase activity towards ISG15. USP18 can efficiently cleave only ISG15 fusions including native ISG15 conjugates linked via isopeptide bonds. Necessary to maintain a critical cellular balance of ISG15-conjugated proteins in both healthy and stressed organisms.

Ubiquitin-conjugating enzyme E2L 6 (UBE2L6) gene (GenBank Accession No. NM_198183 SEQ ID NO: 43; GenBank Accession No. NM_004223 SEQ ID NO: 44) encodes the UBE2L6 protein (GenBank Accession No. NP_937826 SEQ ID NO: 45; GenBank Accession No. NP_004214 SEQ ID NO: 46). The UBE2L6 gene encodes a member of the E2 ubiquitin-conjugating enzyme family. This enzyme is highly similar in primary structure to the enzyme encoded by the UBE2L3 gene. UBE2L6 catalyzes the covalent attachment of ubiquitin or ISG15 to other proteins. UBE2L6 functions in the E6/E6-AP-induced ubiquitination of p53/TP53. It also promotes ubiquitination and subsequent proteasomal degradation of FLT3.

ISG15 ubiquitin-like modifier (ISG15) gene (GenBank Accession No. NM_005101; SEQ ID NO: 47) encodes the ISG15 protein (GenBank Accession No. NM_005101; SEQ ID NO: 48). ISG15 is reported to be an ubiquitin-like protein that is conjugated to intracellular target proteins after IFN-alpha or IFN-beta stimulation. Its enzymatic pathway is partially distinct from that of ubiquitin, differing in substrate specificity and interaction with ligating enzymes. ISG15 conjugation pathway uses a dedicated E1 enzyme, but seems to converge with the ubiquitin conjugation pathway at the level of a specific E2 enzyme. Targets include STAT1, SERPINA3G/SPI2A, JAK1, MAPK3/ERK1, PLCG1, EIF2AK2/PKR, MX1/MxA, and RIG-1. It undergoes deconjugation by USP18/UBP43. It shows specific chemotactic activity towards neutrophils and activates them to induce release of eosinophil chemotactic factors. It was suggested to serve as a trans-acting binding factor directing the association of ligated target proteins to intermediate filaments. Also it may also be involved in autocrine, paracrine and endocrine mechanisms, as in cell-to-cell signaling, possibly partly by inducing IFN-gamma secretion by monocytes and macrophages. It appeaser to display antiviral activity during viral infections In response to IFN-tau, ISG15 was reported to be secreted by the conceptus, may ligate to and regulate proteins involved in the release of prostaglandin F2-alpha (PGF), and thus prevent lysis of the corpus luteum and maintain the pregnancy.

In certain embodiments, in addition to the marker genes of the invention, namely, OAS2, HERC5, UPS18, UBE2L6 and optionally of ISG15, genes, the kits, arrays, compositions and methods of the invention may further include the IFI44L gene. Interferon-induced protein 44-like (IFI44L) gene (GenBank Accession No. NM_0068208; SEQ ID NO: 49) encodes the IFI44L protein (GenBank Accession No. NP_006811; SEQ ID NO: 50) that belongs to the IFI44 family of proteins is located in the cytoplasm and exhibits a low antiviral activity against hepatitis C. The expression of the protein is induced by type I interferon.

In other embodiments, in addition to the marker genes of the invention, namely, OAS2, HERC5, UPS18, UBE2L6 and optionally of ISG15, genes, the kits, arrays, compositions and methods of the invention may further include the RSAD2 gene. Radical S-adenosyl methionine domain containing 2 (RSAD2) gene (GenBank Accession No. NM_080657; SEQ ID NO: 51) encodes the RSAD2 protein (GenBank Accession No. NP_542388; SEQ ID NO:52). RSAD2 is reported to be involved in antiviral defense. It was suggested to impair virus budding by disrupting lipid rafts at the plasma membrane, a feature which is essential for the budding process of many viruses. In addition, it was reported to act through binding with and inactivating FPPS, an enzyme involved in synthesis of cholesterol, farnesylated and geranylated proteins, ubiquinones dolichol and heme. Moreover, it is considered to play a major role in the cell antiviral state induced by type I and type II interferon. Finally, it was reported to display antiviral effect against HIV-1 virus, hepatitis C virus, human cytomegalovirus, and aphaviruses, but not vesiculovirus.

In certain embodiments, in addition to the marker genes of the invention, namely, OAS2, HERC5, UPS18, UBE2L6 and optionally of ISG15, genes, the kits, arrays, compositions and methods of the invention may further include the IFI127 gene. Interferon alpha-inducible protein 27 (IFI27) gene (GenBank Accession Nos. NM_001130080 and NM_005532; SEQ ID NOs:53, 54, respectively) encodes the IFI27 protein (GenBank Accession Nos. NP_001123552 and NP_005523; SEQ ID NOs:55, 56, respectively). The IFI27 protein was reported to promote cell death and mediate IFN-induced apoptosis characterized by a rapid and robust release of cytochrome C from the mitochondria and activation of BAX and caspases 2, 3, 6, 8 and 9.

In yet another embodiment, the kits, arrays, compositions and methods of the invention may further include the STAT1 gene. Signal transducer and activator of transcription 1 (STAT1) gene (GenBank Accession No. NM_007315 SEQ ID NO:57, NM_139266 SEQ ID NO:58) encodes the STAT1 protein (GenBank Accession No. NP_009330 SEQ ID NO:59, NP_644671 SEQ ID NO:60). Signal transducer and transcription activator that mediates cellular responses to interferons (IFNs), cytokine KITLG/SCF and other cytokines and growth factors.

In still another embodiment, the kits, arrays, compositions and methods of the invention may further include the UBE1L gene (GenBank Accession No. NM_003335 SEQ ID NO:61) encodes the protein (GenBank Accession No. NP_003326.2 SEQ ID NO:62). UBE1L, also known as UBA7 (ubiquitin-like modifier activating enzyme 7) activates ubiquitin by first adenylating with ATP its C-terminal glycine residue and thereafter linking this residue to the side chain of a cysteine residue in E1, yielding a ubiquitin-E1 thioester and free AMP.

In still another embodiment, the kits, arrays, compositions and methods of the invention may further include the EIF2AK2 gene. EIF2AK2 eukaryotic translation initiation factor 2-alpha kinase 2 (EIF2AK2) gene (GenBank Accession No. NM_002759.1; SEQ ID NO: 63) encodes the EIF2AK2 protein (GenBank Accession No. NC_000002.11; SEQ ID NO: 64). The protein encoded by this gene is a serine/threonine protein kinase that is activated by autophosphorylation after binding to dsRNA. The activated form of the encoded protein can phosphorylate translation initiation factor EIF2S1, which in turn inhibits protein synthesis. This protein is also activated by manganese ions and heparin. Three transcript variants encoding two different isoforms have been found for this gene.

In accordance with the present invention, the level of expression of the marker genes of the invention is determined in a biological sample of said subject to obtain an expression value.

According to some specific embodiments, the method of the invention involves the determination of the level of expression of OAS2, HERC5, UPS18, UBE216 and optionally of ISG15, genes in a biological sample of the examined subject to obtain an expression value.

In yet further embodiments, the methods of the invention require determining the expression level of the marker genes of the invention, specifically, OAS2, HERC5, UPS18, UBE2L6 and ISG15, genes. It should be appreciated that in certain optional embodiments, in addition to these gene, at least one, at least two, at least three, at least four, at least five or at least six genes of a group consisting of IFI127, RSAD2, STAT1, EIF2AK2, IFI44L and UBE1L genes, as described by the invention may be examined in a biological test sample of a mammalian subject.

Other embodiments of the invention relate to the use of different combinations of the marker genes of the invention, specifically, OAS2, HERC5, UPS18, UBE2L6 and ISG15, genes, with at least one of IFI127, RSAD2, STAT1, EIF2AK2, IFI44L and UBE1L.

In certain embodiments, the kit/s compositions and methods of the invention may involve determining the expression value of control reference genes as described above. Therefore, the kits, compositions and arrays of the invention may comprise detecting molecules as described herein that are specific for at least one of said control reference genes. In certain embodiments, such control reference gene (having an equal expression in samples of responsive and non-responsive subjects) may be a house keeping gene, for example, GAPDH or actin. In other embodiments, the kits, compositions and arrays of the invention may comprise detecting molecules specific for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 and more, specifically, 110, 120, 130, 140, 150, 160, 170, 180, 190, 200 and more, specifically, 300, 350 or 400 genes or control reference genes.

According to specific embodiments, determining the level of expression of the marker genes of the invention, OAS2, HERC5, UPS18, UBE2L6 and optionally of ISG15, genes, in a biological sample of the examined subject may be performed by the step of contacting detecting molecules specific for said marker genes with a biological sample of said subject, or with any nucleic acid or protein product obtained therefrom.

As indicated above, the first step of the diagnostic method of the invention may involve contacting the sample or any aliquot thereof with detecting molecules specific for OAS2, HERC5, UPS18, UBE2L6 and optionally of ISG15, genes.

The term “contacting” means to bring, put, incubate or mix together. As such, a first item is contacted with a second item when the two items are brought or put together, e.g., by touching them to each other or combining them. In the context of the present invention, the term “contacting” includes all measures or steps which allow interaction between the at least one of the detection molecules for OAS2, HERC5, UPS18, UBE2L6 and optionally of ISG15, genes and optionally one suitable control reference gene and the nucleic acid or amino acid molecules of the tested sample. The contacting is performed in a manner so that the detecting molecules of OAS2, HERC5, UPS18, UBE2L6 and optionally of ISG15, genes and of at least one suitable control reference gene can interact with or bind to the nucleic acid molecules or alternatively, a protein product of said marker genes, in the tested sample. The binding will preferably be non-covalent, reversible binding, e.g., binding via salt bridges, hydrogen bonds, hydrophobic interactions or a combination thereof.

In certain embodiments, the detection step further involves detecting a signal from the detecting molecules that correlates with the expression level of said OAS2, HERC5, UPS18, UBE2L6 and optionally of ISG15, genes or product by a suitable means thereof in the sample from the subject. According to some embodiments, the signal detected from the sample by any one of the experimental methods detailed herein below reflects the expression level of said marker genes or product thereof. Such signal-to-expression level data may be calculated and derived from a calibration curve.

Thus, in certain embodiments, the method of the invention may optionally further involve the use of a calibration curve created by detecting a signal for each one of increasing pre-determined concentrations of said OAS2, HERC5, UPS18, UBE2L6 and optionally of ISG15, genes or product thereof. Obtaining such a calibration curve may be indicative to evaluate the range at which the expression levels correlate linearly with the concentrations of said marker genes or products thereof. It should be noted in this connection that at times when no change in expression level of OAS2, HERC5, UPS18, UBE2L6 and optionally of ISG15, genes or product is observed, the calibration curve should be evaluated in order to rule out the possibility that the measured expression level is not exhibiting a saturation type curve, namely a range at which increasing concentrations exhibit the same signal.

It must be appreciated that in certain embodiments such calibration curve as described above may by also part or component in any of the kits provided by the invention herein after.

In other embodiments of the invention, the detecting molecules used for determining the expression levels of the biomarkers of the invention, may be either isolated detecting nucleic acid molecules or isolated detecting amino acid molecules. It should be noted that the invention further encompasses any combination of nucleic and amino acids for use as detecting molecules for the kits, arrays, compositions and methods of the invention. As noted above, in the first step of the method of the invention, the sample or any nucleic acid or protein product derived therefrom is contacted with the detecting molecules of the invention.

In more specific embodiments, for determining the expression level of the biomarkers of the invention, nucleic acid detecting molecule may be used. More specifically, such nucleic acid detecting molecules may comprise isolated oligonucleotides, each oligonucleotide specifically hybridizes to a nucleic acid sequence of an RNA product of said OAS2, HERC5, UPS18, UBE2L6 and optionally of ISG15, marker genes. In an optional embodiment, were the expression level of the biomarkers of the invention are normalized, the method of the invention may use nucleic acid detecting molecules specific for a control reference gene.

According to more specific embodiment, the nucleic acid detecting molecules used by the method of the invention may be at least one of at least one primer, at least one pair of primers, at least one nucleotide probe/s or any combinations thereof.

As used herein, “nucleic acids” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly.

As indicated throughout, in certain embodiments when the detecting molecules used are nucleic acid based molecules, specifically, oligonucleotides. It should be noted that the oligonucleotides used in here specifically hybridize to nucleic acid sequences of the RNA product of the OAS2, HERC5, UPS18, UBE2L6 and optionally of ISG15, marker genes of the invention. Optionally, where also the expression of at least one of IFI127, RSAD2, STAT1, EIF2AK2, IFI44L and UBE1L genes is being examined, the kits, arrays, compositions and method of the invention may use as detecting molecules oligonucleotides that specifically hybridize to a nucleic acid sequence of an RNA product of one of aid at least one of said IFI127, RSAD2, STAT1, EIF2AK2, IFI44L and UBE1L genes. As used herein, the term “hybridize” refers to a process where two complementary nucleic acid strands anneal to each other under appropriately stringent conditions. Hybridizations are typically and preferably conducted with probe-length nucleic acid molecules, for example, 5-100 nucleotides in length, 5-50, 5-40, 5-30 or 5-20.

As used herein “selective or specific hybridization” in the context of this invention refers to a hybridization which occurs between a polynucleotide encompassed by the invention as detecting molecules, and the marker genes of the invention, specifically, the OAS2, HERC5, UPS18, UBE2L6 and optionally of ISG15, genes, and/or any control reference gene, wherein the hybridization is such that the polynucleotide binds to one of said marker genes or to any control reference gene preferentially to any other RNA in the tested sample. In a specific embodiment a polynucleotide which “selectively hybridizes” is one which hybridizes with a selectivity of greater than 60 percent, greater than 70 percent, greater than 80 percent, greater than 90 percent and most preferably on 100 percent (i.e. cross hybridization with other RNA species preferably occurs at less than 40 percent, less than 30 percent, less than 20 percent, less than 10 percent). As would be understood to a person skilled in the art, a detecting polynucleotide which “selectively hybridizes” to OAS2, HERC5, UPS18, UBE2L6 and optionally of ISG15, genes or any control reference gene can be designed taking into account the length and composition.

The terms, “specifically hybridizes”, “specific hybridization” refers to hybridization which occurs when two nucleic acid sequences are substantially complementary (at least about 60 percent complementary over a stretch of at least 5 to 25 nucleotides, preferably at least about 70 percent, 75 percent, 80 percent or 85 percent complementary, more preferably at least about 90 percent complementary, and most preferably, about 95 percent complementary).

The measuring of the expression of the marker genes of the invention, specifically, OAS2, HERC5, UPS18, UBE2L6 and optionally of ISG15, genes and any control reference gene and combination thereof can be done by using those polynucleotides as detecting molecules, which are specific and/or selective for said marker genes or any control reference gene to quantitate the expression of said genes or any control reference gene. In a specific embodiment of the invention, the polynucleotides which are specific and/or selective for said marker genes or any control reference gene may be probes or a pair of primers. It should be further appreciated that the methods, as well as the compositions and kits of the invention may comprise, as an oligonucleotide-based detection molecule, both primer/s and probe/s or any combination thereof.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe is at least 5 or preferably, 8 nucleotides in length and less than the length of a complete mRNA (cDNA). A probe may be 5, 6, 7, 8, 9, 10, 11, 12, 13, 14, 15, 16, 17, 18, 19, 20, 21, 22, 23, 24, 25, 26, 27, 28, 29 and up to 30 nucleotides in length as long as it is less than the full length of the target mRNA or any gene encoding said mRNA (cDNA). Probes can include oligonucleotides modified so as to have a tag which is detectable by fluorescence, chemiluminescence and the like. The probe can also be modified so as to have both a detectable tag and a quencher molecule, for example TaqMan® and Molecular Beacon® probes.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′0-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

Thus, according to one embodiment, such oligonucleotides are any one of at least one primer, at least one pair of primers, at least one nucleotide probe/s or any combination thereof, and wherein the level of expression of marker genes of the invention, OAS2, HERC5, UPS18, UBE2L6 and optionally of ISG15, is determined using a nucleic acid amplification assay selected from the group consisting of: a Real-Time PCR, micro array, PCR, in situ hybridization and comparative genomic hybridization.

The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan®, MGB-TaqMan®), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan® and MGB-TaqMan® the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified OAS2, HERC5, UPS18, UBE2L6 and optionally of ISG15, genes or any control reference gene produced in a PCR amplification reaction. During PCR, the amplified products created using suitable primers hybridize to probe nucleic acids (TaqMan® probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR. The method and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan® (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan® system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan® and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons® which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or part of said OAS2, HERC5, UPS18, UBE2L6 and optionally of ISG15, genes or any control reference gene. More particularly, it is important to choose regions which will permit hybridization to the target nucleic acids. Factors such as the Tm of the oligonucleotide, the percent GC content, the degree of secondary structure and the length of nucleic acid are important factors.

It should be further noted that a standard Northern blot assay can also be used to ascertain an RNA transcript size and the relative amounts of said OAS2, HERC5, UPS18, UBE2L6 and optionally of ISG15, genes or of any control gene product, in accordance with conventional Northern hybridization techniques known to those persons of ordinary skill in the art.

Particular embodiments of the method of the invention are based on detecting the expression values of said OAS2, HERC5, UPS18, UBE2L6 and optionally of ISG15, genes. According to this embodiment, the detecting nucleic acid molecules used by the method of the invention comprise isolated oligonucleotides that specifically hybridize to a nucleic acid sequence of an RNA product of one of said marker genes, and isolated oligonucleotides that specifically hybridize to a nucleic acid sequence of at least one of the control reference gene.

It should be appreciated that all the detecting molecules used by any of the kits, arrays, compositions and methods of the invention described herein, are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95 percent pure) of non-nucleotide material naturally associated with it, and thus is distinguished from isolated chromosomes.

Still further, it must be understood that any of the detecting molecules (for example, primers and/or probes) or reagents used by the compositions, kits, arrays and in any step of the methods of the invention are non-naturally occurring products or compounds, As such, none of the detecting molecules of the invention are directed to naturally occurring compounds or products.

The invention further contemplates the use of amino acid based molecules such as proteins or polypeptides as detecting molecules disclosed herein and would be known by a person skilled in the art to measure the protein products of the marker genes of the invention, specifically, OAS2, HERC5, UPS18, UBE2L6 and optionally of ISG15, genes. Techniques known to persons skilled in the art (for example, techniques such as Western Blotting, Immunoprecipitation, ELISAs, protein microarray analysis, Flow cytometry and the like) can then be used to measure the level of protein products corresponding to the biomarkers of the invention. As would be understood to a person skilled in the art, the measure of the level of expression of the protein products of the biomarkers of the invention, specifically, OAS2, HERC5, UPS18, UBE2L6 and optionally of ISG15, genes, requires a protein, which specifically and/or selectively binds to the biomarker genes of the invention.

As indicated above, the detecting molecules of the invention may be amino acid based molecules that may be referred to as protein/s or polypeptide/s. As used herein, the terms “protein” and ‘polypeptide’ are used interchangeably to refer to a chain of amino acids linked together by peptide bonds.

In specific embodiments, the detecting amino acid molecules are isolated antibodies, with specific binding selectively to the proteins encoded by OAS2, HERC5, UPS18, UBE2L6 and optionally of ISG15, genes as detailed above. Using these antibodies, the level of expression of proteins encoded by said marker genes may be determined using an immunoassay which is selected from the group consisting of FACS, a Western blot, an ELISA, a RIA, a slot blot, a dot blot, immunohistochemical assay and a radio-imaging assay.

The term “antibody” as used in this invention includes whole antibody molecules as well as functional fragments thereof, such as Fab, F(ab′)2, and Fv that are capable of binding with antigenic portions of the target polypeptide, i.e. proteins encoded by said OAS2, HERC5, UPS18, UBE2L6 and optionally of ISG15, genes. The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which as used herein refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, 1gM or subtypes thereof. The antibody can be conjugated to a functional moiety (e.g., a compound which has a biological or chemical function. The antibody used by the invention interacts with a polypeptide that is a product of OAS2, HERC5, UPS18, UBE2L6 and optionally of ISG15, genes, with high affinity and specificity.

As noted above, the term “antibody” also encompasses antigen-binding fragments of an antibody, for example, Fab, Fab′, (Fab′)2, Fv, Single chain antibody (“SCA”, or ScFv), or any combination thereof.

Methods of generating such antibody fragments are well known in the art (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immuno-affinity chromatography as well as gel filtration, zone electrophoresis, etc.

Still further, for diagnostic and monitoring uses described herein after, the antibodies specific for the proteins encoded by OAS2, HERC5, UPS18, UBE2L6 and optionally of ISG15, used by the present invention may optionally be covalently or non-covalently linked to a detectable label. The term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody. More specifically, detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA and competitive ELISA and other similar methods known in the art) and colorimetric labels such as colloidal gold or colored glass or plastic (e.g. polystyrene, polypropylene, latex, etc.) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

More particularly, “selectively bind” in the context of proteins encompassed by the invention refers to the specific interaction of a any two of a peptide, a protein, a polypeptide an antibody, wherein the interaction preferentially occurs as between any two of a peptide, protein, polypeptide and antibody preferentially as compared with any other peptide, protein, polypeptide and antibody.

Thus, under designated immunoassay conditions, the specified antibodies bind to a particular epitope at least two times the background and more typically more than 10 to 100 times background. More specifically, “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

According to one embodiment, where amino acid-based detection molecules are used, the expression level of the proteins encoded by OAS2, HERC5, UPS18, UBE2L6 and optionally of ISG15, genes, in the tested sample can be determined using different methods known in the art, specifically method disclosed herein below as non-limiting examples.

Still further, according to certain embodiments, the method of the invention uses any appropriate biological sample. The term “biological sample” in the present specification and claims is meant to include samples obtained from a mammal subject.

It should be recognized that in certain embodiments a biological sample may be for example, blood cells, blood, biopsies of organs or tissues, bone marrow, lymph fluid, serum, plasma, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. More specific embodiments, the sample may be any one of peripheral blood mononuclear cells and biopsies of organs or tissues.

According to an embodiment of the invention, the sample is a cell sample. More specifically, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to a specific embodiment, the sample used by the method of the invention is a sample of peripheral blood mononuclear cells (PBMCs).

The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) bio-markers of the invention.

It should be appreciated that any subclass of blood cells may be used for the method of the invention, in more specific embodiments the blood sample may comprise CD⁺14 cells.

As show in Example 1, examination of blood samples of HCV patients with the marker genes of the invention gave the best predictive results using OAS2, HERC5, UPS18, UBE2L6. In other embodiments OAS2, HERC5, UPS18, UBE2L6 and optionally ISG15 genes may be used as markers.

Thus, in certain embodiments, the sample is a blood sample, and the method of the invention comprises determining the level of expression of OAS2, HERC5, UPS18, UBE2L6 and optionally of ISG15, genes in said blood sample. In yet another embodiment, in case of blood sample, the method of the invention may use OAS2, HERC5, UPS18 and UBE2L6 as markers. Still further, in another embodiment, the method of the invention comprises determining the level of expression of OAS2, HERC5, UPS18, UBE2L6 and ISG15, genes in said blood sample.

In yet another embodiment, the sample may be a biopsy of human organs or tissue, specifically, liver biopsy. According to such embodiments, the method of the invention comprises determining the level of expression of OAS2, HERC5, UPS18, UBE2L6 and of ISG15, genes in said liver tissue sample.

According to some embodiments, the sample may be biopsies of organs or tissues. The biopsies may be obtained by a surgical operation from an organ or tissue of interest, for example liver biopsy, cerebrospinal fluid (CSF), brain biopsy, skin biopsy.

The term biopsy used herein refers to a medical test commonly performed by a surgeon or an interventional radiologist involving sampling of cells or tissues for examination. It is the medical removal of tissue from a living subject to determine the presence or extent of a disease. The tissue is generally examined under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

According to some embodiments of the invention, the cell is a liver cell.

It should be noted that liver cells (hepatic cell) can be obtained by a liver biopsy (e.g., using a surgical tool or a needle). It should be noted that certain embodiments of the invention contemplate the use of different biological samples.

The invention further encompasses the use of the biomarkers of the invention as a biomarker for predicting, assessing and monitoring response to interferon treatment in subjects in need of interferon treatment. Such subject may be for example a subject suffering from an immune-related disorder.

It should be noted that an “Immune-related disorder” is a condition that is associated with the immune system of a subject, either through activation or inhibition of the immune system, or that can be treated, prevented or diagnosed by targeting a certain component of the immune response in a subject, such as the adaptive or innate immune response.

In specific embodiments, such immune-related disorder may be any one of an infectious condition, an autoimmune disease and a proliferative disorder.

In yet another embodiment, the method of the invention may be suitable for predicting responsiveness to interferon treatment in a subject suffering from an inflammatory disorder, specifically, an infectious condition caused by a pathogenic agent. More specifically, such infectious conditions may be any one of viral diseases, protozoan diseases, bacterial diseases, parasitic diseases, fungal diseases and mycoplasma diseases.

It should be appreciated that an infectious disease as used herein also encompasses any infectious disease caused by a pathogenic agent. Pathogenic agents include prokaryotic microorganisms, lower eukaryotic microorganisms, complex eukaryotic organisms, viruses, fungi, prions, parasites and yeasts.

A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

The term parasite includes, but not limited to, infections caused by somatic tapeworms, blood flukes, tissue roundworms, ameba, and Plasmodium, Trypanosoma, Leishmania, and Toxoplasma species.

In certain embodiments, the kits, arrays, compositions and methods of the invention are applicable for a subject suffering from an infectious disease.

The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, hepatitis A, poliovirus, rubella virus, hepatitis C, arboviruses, rabies virus, influenza viruses A and B, measles virus, mumps virus, HIV, HTLV I and II.

In certain embodiments, the kits, arrays, compositions and methods of the invention are applicable for and assessing responsiveness of a mammalian subject to interferon treatment of a subject suffering from an infectious disease caused by any one of hepatitis C, A or B virus (HCV, HAV, HBV), HIV, influenza (specifically, H1N1 and H5N1), dengue virus, West Nile virus (WNV), Polio virus. In more specific embodiments the subject is suffering from an HCV infection.

As shown by the Examples, the biomarkers used by method of the invention distinguish between interferon responders and non-responders HCV infected subjects. Therefore, the Kits, arrays, compositions and methods of the invention may be used for predicting interferon responsiveness in subjects suffering from viral infections, for example, Hepatitis C virus infection (type 1, 2, 3 or 4) infections.

As used herein the term “HCV” refers to hepatitis C virus having genotype 1 (also known as HCV Type 1), genotype 2 (also known as HCV Type 2), genotype 3 (also known as HCV Type 3), genotype 4 (also known as HCV Type 4), genotype 5 (also known as HCV Type 5) or genotype 6 (also known as HCV Type 6).

The phrase “HCV infection” encompasses acute (refers to the first 6 months after infection) and chronic (refers to infection with hepatitis C virus which persists more than 6 month) infection with the hepatitis C virus. Thus, according to some embodiments of the invention, the subject is diagnosed with chronic HCV infection.

According to some embodiments of the invention, the subject is infected with HCV type 1. According to some embodiments of the invention, the subject is infected with HCV type 2, 3 or 4.

More specifically, Hepatitis C virus (HCV or sometimes HVC) is a small (55-65 nm in size), enveloped, positive-sense single-stranded RNA virus of the family Flaviviridae and as indicated herein, is the cause of hepatitis C in humans. The hepatitis C virus particle consists of a core of RNA, surrounded by an icosahedral protective shell of protein, and further encased in a lipid (fatty) envelope of cellular origin. The Hepatitis C virus has a positive sense single-stranded RNA genome consisting of a single open reading frame that is 9600 nucleotide bases long.

Hepatitis C is an infectious disease affecting primarily the liver, is caused by the hepatitis C virus (HCV). The infection is often asymptomatic, but chronic infection can lead to scarring of the liver and ultimately to cirrhosis, which is generally apparent after many years. In some cases, those with cirrhosis will go on to develop liver failure, liver cancer, or life-threatening esophageal and gastric varices. The invention in some embodiments thereof provides methods, kits and compositions for predicting responsiveness of HCV patients to treatment, specifically, interferon.

In yet other embodiments, it should be appreciated that the Kits, arrays, compositions and methods of the invention may be applicable for predicting responsiveness for interferon treatment in subjects suffering from influenza infections. Thus, in specific embodiments, the infectious condition is a virus of the Orthomyxoviridae, family, such as, but not limited to, Influenza virus A, Influenza virus B, Influenza virus C or any subtype and reassortants thereof.

As used herein the term Influenza viruses refers to orthomyxoviruses, and fall into three types; A, B and C. Influenza A and B virus particles contain a genome of negative sense, single-strand RNA divided into 8 linear segments. Co-infection of a single host with two different influenza viruses may result in the generation of reassortant progeny viruses having a new combination of genome segments, derived from each of the parental viruses. Influenza A viruses have been responsible for four recent pandemics of severe human respiratory illness.

The kits, arrays, compositions and methods of the invention may further applicable for predicting responsiveness to interferon, of a subject suffering from an immune-related disorder. Non-limiting examples for autoimmune disorders include Multiple Sclerosis (MS), inflammatory arthritis, rheumatoid arthritis (RA), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, Behcet's syndrome, ankylosing spondylitis, pemphigus, bullous pemphigoid, dermatitis herpetiformis, inflammatory bowel disease, ulcerative colitis and Crohn's disease and fatty liver disease.

In more specific embodiment, the kits, arrays, compositions and methods of the invention may be applicable for subjects suffering from MS. As used herein the phrase “multiple sclerosis” (abbreviated MS, formerly known as disseminated sclerosis or encephalomyelitis disseminata) is a chronic, inflammatory, demyelinating disease that affects the central nervous system (CNS). Disease onset usually occurs in young adults, is more common in women, and has a prevalence that ranges between 2 and 150 per 100,000 depending on the country or specific population.

MS is characterized by presence of at least two neurological attacks affecting the central nervous system (CNS) and accompanied by demyelinating lesions on brain magnetic resonance imaging (MRI). MS takes several forms, with new symptoms occurring either in discrete episodes (relapsing forms) or slowly accumulating over time (progressive forms). Most people are first diagnosed with relapsing-remitting MS (RRMS) but develop secondary-progressive MS (SPMS) after a number of years. Between episodes or attacks, symptoms may go away completely, but permanent neurological problems often persist, especially as the disease advances.

The invention further encompasses the use of OAS2, HERC5, UPS18, UBE2L6 and optionally of ISG15, genes as biomarkers for predicting, assessing and monitoring the response to interferon treatment in subjects suffering from any condition related to the conditions described above. It is understood that the interchangeably used terms ‘linked”, “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition” and the like, as they relate to a subject's health, are used interchangeably and have meanings ascribed to each and all of such terms.

As explained in the Examples, the inventors have analyzed the expression values of OAS2, HERC5, UPS18, UBE2L6 and optionally of ISG15, genes and found that changes in the expression level of the above are indicative of an increased likelihood for respond to interferon treatment and to be in a relapse stage.

As indicated herein before, the kits, arrays and compositions of the invention described herein before, are particularly intended for predicting assessing and monitoring response to interferon treatment in a subject suffering from a disease treated with interferon.

In certain embodiments, the prognostic the kits, arrays and compositions of the invention are particularly suitable for use according to the prognostic method of the invention.

Thus, the invention further provides kits, arrays and compositions for use in the prognosis of disease treated with interferon as well as monitoring and predicting responsiveness to interferon treatment and early diagnosis of relapse.

It should be appreciated that the composition of the invention may be used for predicating response of a mammalian subject to interferon treatment. According to one embodiment of the composition of the invention, the kits, arrays and compositions may be used to perform the prognostic method of the invention using a test sample of the subject obtained during diagnosis of a disease. The expression value of the biomarkers, OAS2, HERC5, UPS18, UBE2L6 and optionally of ISG15, genes obtained from the examined sample is compared to a predetermined standard expression value or cutoff value. A positive expression value, or in other words, a higher expression value of the biomarkers of the invention, as compared to the predetermined standard expression value (cutoff value), indicates that said subject belongs to a pre-established population associated with lack of responsiveness to interferon treatment and therefore, the subject may be considered as a non-responsive subject.

It should be appreciated that the kits, arrays and compositions of the invention may be used for assessing responsiveness of a mammalian subject to interferon treatment or evaluating the efficacy of interferon treatment on a subject and for diagnosis of relapse.

Furthermore, in another embodiment of the kits, arrays and compositions of the invention, the composition may be used according to the prognostic method of the invention using at least two test samples of the subject, preferably three or more samples, wherein the samples are collected at different times from the subject.

The at least two time points are adjusted such that the required information is obtained. For example, in order to asses responsiveness to treatment, the first time point is before initiation of treatment and the second time point is at any time after initiation of treatment.

For example, in order to determine relapse, the at least two time points are obtained after initiation of treatment, preferably one of the time points is at remission.

The rate of change of the normalized expression values of the marker genes of the invention, specifically, the OAS2, HERC5, UPS18, UBE2L6 and optionally of ISG15, genes between said temporally-separated test samples is being calculated.

The kits, arrays and compositions of the invention may therefore facilitate the prediction of probability of a patient to respond to interferon treatment, the monitoring and early sub-symptomatic diagnosis or prediction of a relapse in a subject when used according to the method of the invention for analysis of more than a single sample along the time-course of diagnosis, treatment and follow-up.

It should be appreciated that the invention may provide a method for treating, preventing, ameliorating or delaying the onset of an immune-related disorder in a subject. More specifically, the method of the invention may comprise the step of: (a) predicting, assessing and monitoring responsiveness of the tested subject to interferon treatment using the kits, arrays, compositions and methods of the invention. In a further step (b), selecting an interferon treatment regimen based on said responsiveness thereby treating said subject.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment and diagnosis methods herein described is desired, including humans. More specifically, the composition of the invention is intended for mammals. By “mammalian subject” is meant any mammal for which the proposed therapy is desired, including human, equine, canine, and feline subjects, most specifically humans.

Still further, it must be appreciated that the invention further provides prognostic methods comprising the step of (a) providing a composition comprising detecting molecules specific for determining the level of expression of OAS2, HERC5, UPS18, UBE2L6 and optionally of ISG15 genes and a biological sample, specifically, a sample obtained from a subject to be diagnosed; (b) determining the level of expression of a group of genes comprising OAS2, HERC5, UPS18, UBE2L6 and optionally of ISG15 in the composition, to obtain an expression value for each of said genes; and (c) determining if the expression value obtained in step (b) is any one of positive or negative with respect to a predetermined standard expression value or to an expression value of said genes in at least one control sample; thereby predicting, assessing and monitoring responsiveness of a mammalian subject to said treatment regimen.

In yet some further embodiments, where a dynamic method is applied, the invention provides prognostic methods comprising the steps of: (a) providing at least two compositions comprising detecting molecules specific for determining the level of expression of OAS2, HERC5, UPS18, UBE2L6 and optionally of ISG15 genes and a biological sample obtained from a subject to be diagnosed. It should be noted that the biological samples comprised within the compositions used by the method of the invention are temporally-separated samples. In a similar manner, when control samples are used, the method of the invention involves the step of providing similar compositions comprising the detecting molecules specific for the marker genes of the invention and the control samples.

The next step (b) involves determining the level of expression of said genes, in said at least one composition provided, to obtain an expression value for each of said genes or sum thereof; and (c) repeating steps (b) to obtain an expression value of said group of genes for at least one more composition comprising said temporally-separated sample; (d) calculating the rate of change of the expression value of said genes between said compositions; (e) calculating the sum of said rate of change in the expression of said genes as determined in step (d) to obtain a Sum rate of change value; and (f) determining if the Sum rate of change value of said genes obtained in step (e) is positive or negative with respect to a predetermined standard Sum rate of change value or to a Sum rate of change value calculated for said genes in at least one control composition; thereby monitoring disease progression or providing an early prognosis for disease relapse.

Still further, it must be understood that in certain embodiments, the invention further provides a prognostic composition comprising (a) detecting molecules specific for determining the level of expression of OAS2, HERC5, UPS18, UBE2L6 and optionally of ISG15 genes and (b) a biological sample. In certain embodiments, the biological sample may be obtained from the subject that is to be prognosed. In some embodiments, the sample may be a control sample, as discussed herein before. In an optional embodiment, the detecting molecules may be attached to a solid support. As such, the composition of the invention may be specifically suitable for performing any of the prognostic methods disclosed by the invention.

The term “treatment or prevention” refers to the complete range of therapeutically positive effects of administrating to a subject including inhibition, reduction of, alleviation of, and relief from, a condition known to be treated with interferon, for example an immune-related disorder as detailed herein. More specifically, treatment or prevention of relapse or recurrence of the disease includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

All scientific and technical terms used herein have meanings commonly used in the art unless otherwise specified. The definitions provided herein are to facilitate understanding of certain terms used frequently herein and are not meant to limit the scope of the present disclosure. As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

As used herein the term “about” refers to ±10%. The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “about” as used herein indicates values that may deviate up to 1 percent, more specifically 5 percent, more specifically 10 percent, more specifically 15 percent, and in some cases up to 20 percent higher or lower than the value referred to, the deviation range including integer values, and, if applicable, non-integer values as well, constituting a continuous range. It must be noted that, as used in this specification and the appended claims, the singular forms “a”, “an” and “the” include plural referents unless the content clearly dictates otherwise.

### EXAMPLES

The results shown below provide genetic markers that may serve as markers for predicting the response of a subject in need to interferon treatment before initiation of treatment. Therefore, the present invention exemplifies the feasibility of using these five specific marker genes in personalized medicine.

Specifically, the Examples shown here demonstrate the ability to predict response to IFN treatment in HCV patients before initiation of treatment using both blood samples and liver biopsies.

### Patients

The patients recruited for this study must consent to be in the study and must have signed an approved consent form conforming to institutional guidelines. In addition, the patients must satisfy all of the following criteria:
Patient age 18-70 years
Patients must have Hepatitis C type 1.

### Conditions for Patient Ineligibility

Patients with Hepatitis A or B.
HIV positive patients

### Data on HCV Patients in Liver Biopsy as Well as for the Blood Samples Study

### Conditions for Patient Eligibility

Men and Women who satisfy all of the following criteria are the only patients who will be eligible for this study:
Patient age 18-70 years
The patient must consent to be in the study and must have signed an approved consent form conforming to institutional guidelines.
Patients must have Hepatitis C type 1.
Patients who were scheduled for liver biopsy as standard of care procedure.

### Conditions for Patient Ineligibility

Patients with Hepatitis A or B.
HIV positive patients
Patient with HCC
Heavy alcohol users

Interferon treatment is according to the guidelines of the Israel Liver specialist and was not changed due to the viral load test.

### Experimental Procedures

Isolation of Total RNA from Blood and Liver Tissue

Liver biopsy—Small portion of the biopsy (3-4 mm) is stored in Trizol at −70° C. till the extraction of RNA. The liver biopsy is thawed up prior to extraction and is grinded by homogenizer. The extraction is done by commercial kit of Life technologies such as RNAqueous Kit 50 cartridges/PC AB_AM1912. The quality and quantity of RNA is measure by NanoDrop and Bioanalyzer.

Blood samples—From the whole blood sample a fraction of PBMC (peripheral blood monocytes cells is separated by using Ficoll-Hypaque gradient. RNA Later (about 0.3 ml) is to cells pellet and transfer to 2-8° C. for 24 hours. After 24 hours transfer the cells sample at −70° C.

The PBMC sample is thawed up prior to extraction and is grinded by homogenizer. The extraction is done by commercial kit of Life technologies such as RNAqueous Kit 50 cartridges/PC AB_AM1912. The quality and quantity of RNA is measure by NanoDrop and Bioanalyzer.

### Conditions and Procedure for RT-PCR

RT PCR measurements were performed using ABI 7900HT Sequence Detection System or AB StepOnePlus Real-Time PCR system.

Mixture preparation: sample cDNA is diluted to 25 ng/μl. Into 19 μl of Master mix and water 1 μl of sample cDNA is. Each sample is measured in triplicates against each primer (gene probe) and additional normalizing gene. There is standard run cycle for default Taqman profile:

40 cycles

| | | Enzyme | | Anneal/ |
|---|---|---|---|---|
| | | activation | Denature | extend |
| Temperature ° C. | 50 | 95 | 95 | 60 |
| Time | 2 min | 10 min | 15 sec | 1 min |

### IFN Treatment of the Examined Patients

Patients are treated according SOC (standard of care) guideline of the Israel Society for Liver Research 1012. The treatment include double therapy of Pegylated interferon alpha (peg IFNα-2a/2b and Ribavirin (RBV) for 24-48 weeks. peg IFNα is injected once a week and RBV pills are taken on daily base.

### Measurements of Virus Load

### Quantitative Viral Load Tests

These tests measure the amount of virus in one milliliter of blood and are based of RT PCR technology. The most used are the kits of Roche Molecular Diagnostic such as: COBAS® TaqMan® HCV Test v2.0-Hepatitis C virus (HCV) viral load quantification in human serum or plasma measures the RNA levels or COBAS® AmpliPrep/COBAS® TaqMan® HCV Test —An in vitro nucleic acid amplification test for the quantitation of HCV RNA in human plasma or serum.

### Example 1

### Prediction of Response to Treatment of IFN-α in Blood Samples of HCV Patients

Analysis of the genetic profile in Peripheral Blood Mononucleated Cell (PBMC) of HCV patients was done on samples obtained before initiation of IFN-α treatment. These analyses revealed the important role of a group of genes that may serve as a predictive tool to predict response to treatment before initiation of treatment.

The expression levels of the following genes: UBE2L6, USP18, HERC5, OAS2 and ISG15 (using 3 probes) in each patient was measured by RT-PCR and normalized to a control gene GAPDH.

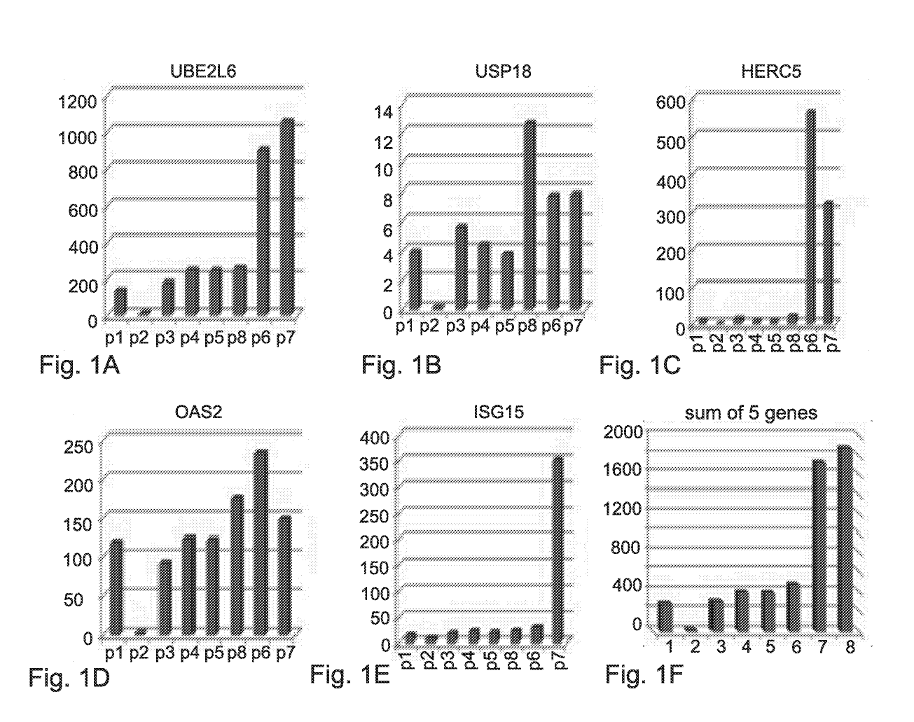

FIGS. 1A to 1E show the expression levels of these genes in blood samples of eight HCV patients.

The normalized expression of each gene was scaled according to the Formula (I):

(expression−min)/(max−min). The scaled expression was within values of 0 to 1.

The “Expression” in Formula (I) refers to the expression of each gene in each one of the patients, wherein the “min” and “max” values represent the min and max expression value of each gene within the cohort of patients.

Then, the sum of the normalized and scaled expression of the five genes in each one of the tested patients was calculated (FIG. 1F).

As can be seen from FIG. 1F, the sum of the normalized and scaled expression of the five tested genes: UBE2L6, USP18, HERC5, OAS2 and ISG15 was found to be significantly lower in six of the eight patients (denoted as p2, p1, p3, p5, p4, and p8) compared to the other two patients (denoted as p6 and p7).

In addition, in each one of the eight patients, the virus load was studies before treatment and 4 weeks after treatment with IFN-α. The viral load was tested using commercial kits as described in the experimental procedures.

Based on the results of the change in virus load measured before treatment and after 4 weeks of treatment, two populations of HCV patients were defined: responders and non-responders.

A responder was considered as a patient that the amount of viral load was reduced by more than 100 within 4 weeks, (2 in log 10). A non-responder was considered as a patient that the amount of viral load was reduced by less than 100 within 4 weeks, (2 in log 10).

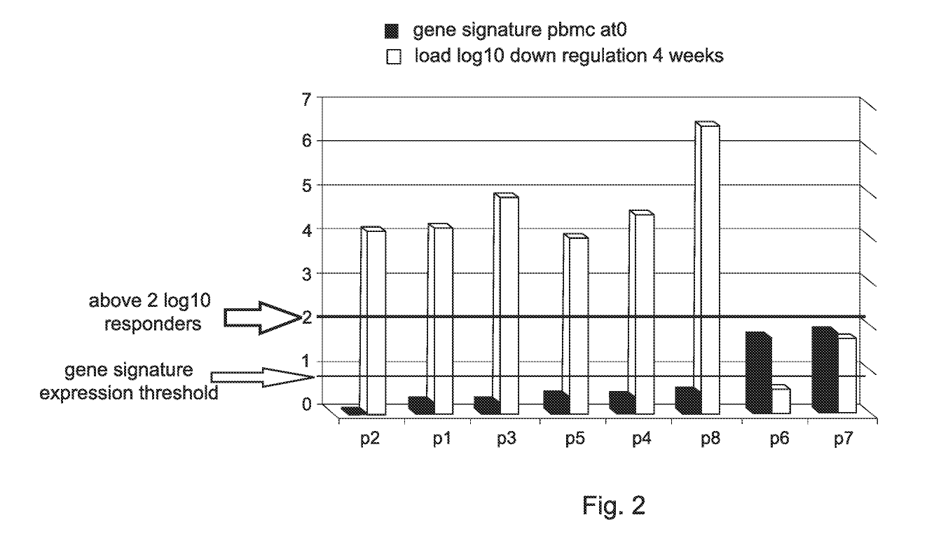

As can be seen in FIG. 2, the patients denoted as p2, p1, p3, p5, p4, and p8 experienced an amount of down regulation of virus load higher than 100 (observed as 2 in log 10 scale) and are thus considered responders to IFN-α treatment in line with the definition above.

In contrast, patients denoted as p6 and p7 experienced an amount of down regulation of virus load lower than 100 (observed as 2 in log 10 scale) and are thus considered non-responders to IFN-α treatment in line with the definition above.

The results in FIG. 2 demonstrated that the sum of normalized and scaled expression of the five genes UBE2L6, USP18, HERC5, OAS2 and ISG15 was significantly reduced in patients that were considered as responders (p2, p1, p3, p5, p4, and p8) compared to the expression in patients considered as non-responders (p6 and p7).

Based on these results, it can be concluded that experimental data obtained before initiation of treatment from blood samples of HCV patients by a routine method such as RT-PCR may accurately predict if the patient will respond to treatment based on the expression level of these five genes.

For example, when using a set of five genes, it is possible to select threshold value of the sum to be about 0.5. Then, a patient having a higher sum of these five genes would be predicted to be a non-responder.

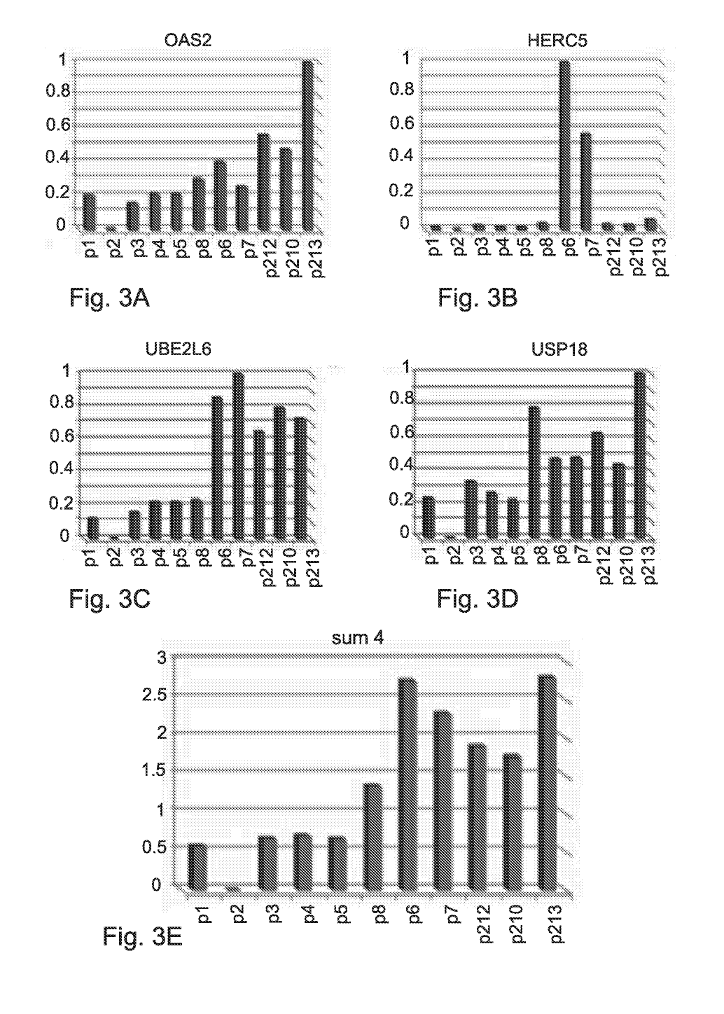

In addition, experimental RT-PCR data of four genes HERC5, OAS2, UBE2L6 and USP18 obtained from blood of eleven HCV patients was tested. The difference in the normalized and scaled expression of four genes measured in PBMC of eight HCV patients before initiation of treatment (day “0”) is shown in FIGS. 3A to 3D.

FIG. 3E shows the sum of the normalized and scaled expression of the four genes, HERC5, OAS2, UBE2L6 and USP18. As can be seen, the sum was found to be higher in the patients considered as non-responsive.

For example, when using a set of four genes, it is possible to select threshold value of the sum to be about 1.5. Then, a patient having a higher sum of these five genes would be predicted to be a non-responder.

The results shown herein demonstrate the importance of determining the expression of a minimal set of four genes HERC5, OAS2, UBE2L6 and USP18 or five gens HERC5, OAS2, UBE2L6 USP18 and ISG15 in predicting response to treatment.

### Example 2

### Prediction of Response to Treatment of IFN-α in Liver Samples of HCV Patients

Analysis of the genetic profile in obtained from liver biopsy of 18 HCV patients was done before initiation of treatment. These analyses have revealed the important role of a group of genes that can serve as a predictive tool both to predict response to treatment before initiation of the treatment.

The expression of the following nine genes OAS2, HERC5, USP18, UBE2L6, ISG15, IFI27, IFI44L, UBE1L and IFIH1 was determined in liver biopsies of HCV patients before initiation of treatment, using RT-PCR.

The expression of each gene (3 probes) was measured using RT-PCR and normalize to the expression of a control gene in each patient GAPDH.

Then, the normalized expression of each gene was scaled according to the Formula (I):

(expression−min)/(max−min). The scaled expression was within values of 0 to 1.

The “Expression” in Formula (I) refers to the expression of each gene in each one of the patients, wherein the “min” and “max” values represent the min and max expression value of each gene within the cohort of patients.

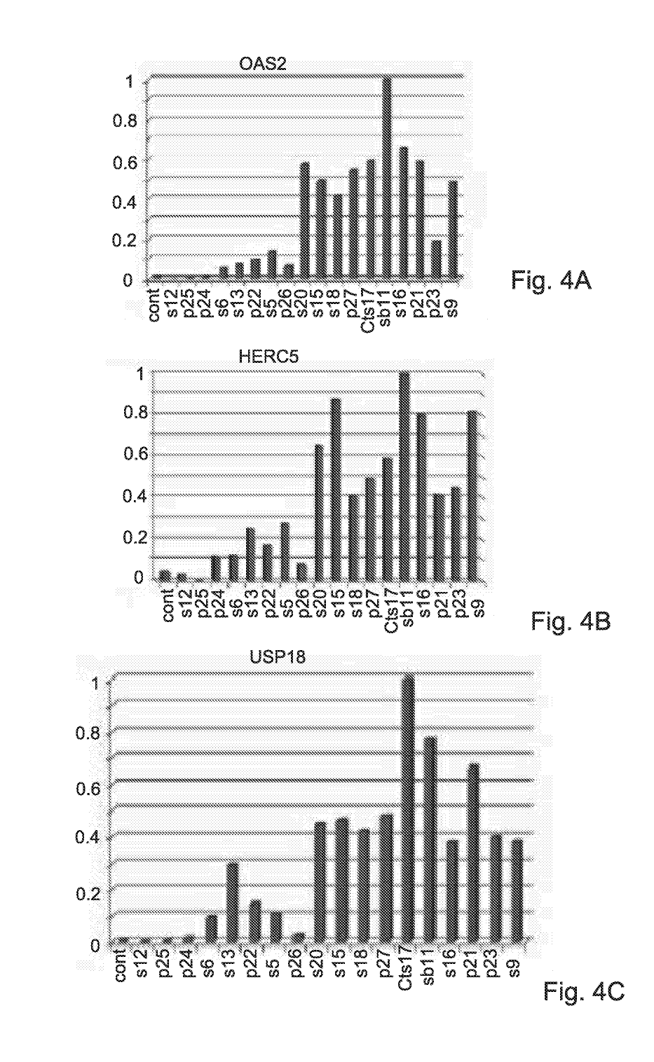

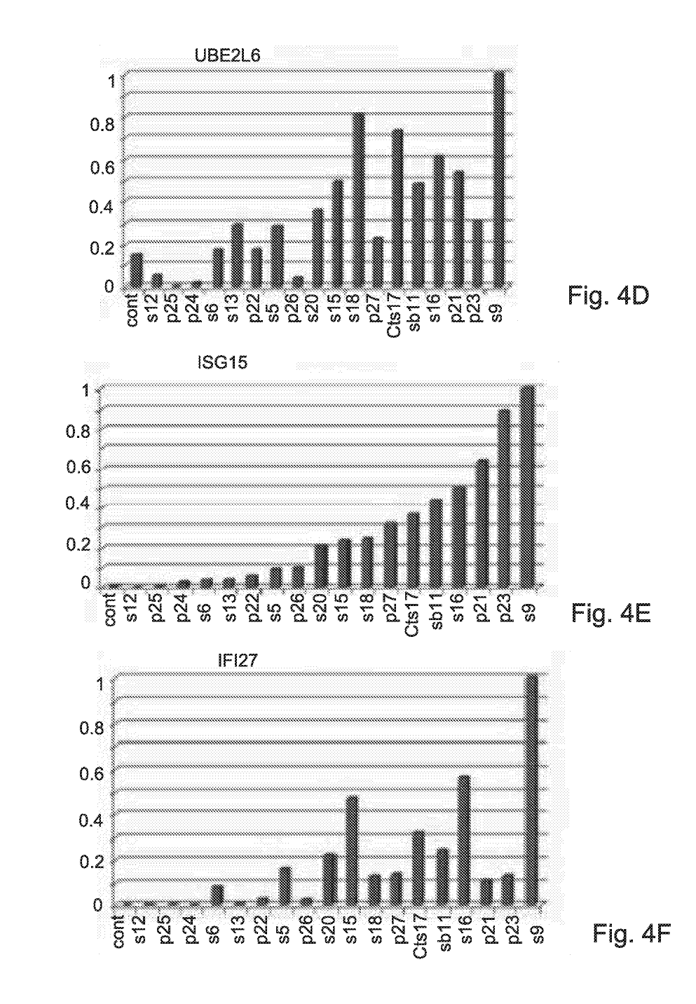

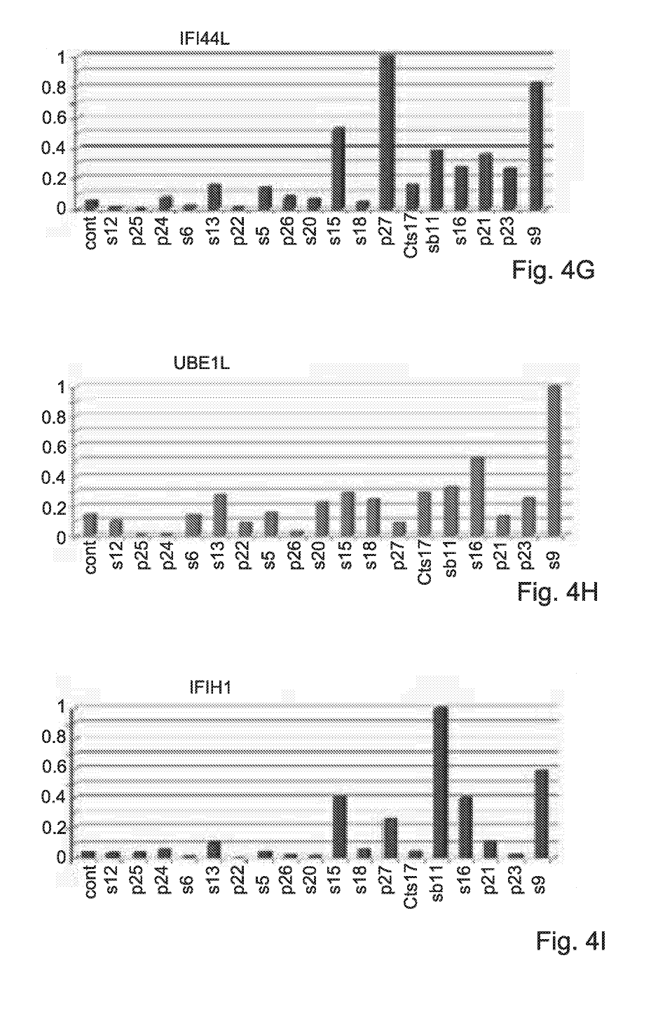

The difference in the genetic expression measured before initiation of treatment (day “0”) between responders and non-responders can be viewed from FIG. 4. The expression of five OAS2, HERC5, USP18, UBE2L6 and ISG15 genes was found to be significantly lower in patients that are considered as responders (s12, p25, p24, s6, s13, p22, s5, p26) compared to the expression in patients considered as non-responders (s20, s15, s18, p27, cts17, sb11, s16, p21, p23, s9).

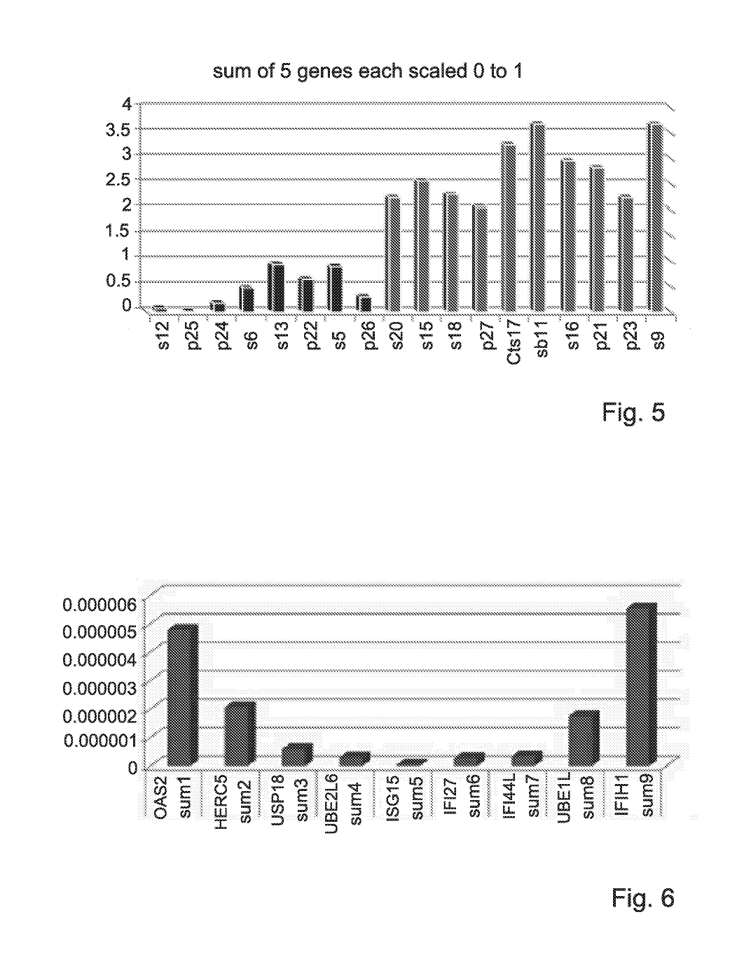

The sum of the normalized and scaled expression of the five genes: OAS2, HERC5, USP18, UBE2L6 and ISG15 in each one of the tested patients is shown in FIG. 5. A difference in the sum of these five genes between was observed responders and non-responders, with the sum being higher in non-responders.

In order to asses a minimal set of genes, the normalized scaled expression of each one of the genes was summed in each one of the tested patients starting from one gene to a final sum of nine genes as detailed in Table 1.

Each one of the nine sums was tested for the ability to predict responsiveness for the group of tested patients. Table 1 shows the sum of genes determined and the corresponding p-value obtained in t-test analysis for the ability to predict responsiveness.

TABLE 1
the sum of the genes and the corresponding p-values

| Sum # | Sum of genes | p-value |
|---|---|---|
| 1 | OAS2 | 4.78e⁻⁶ |
| 2 | OAS2 and HERC5 | 2.06e⁻⁶ |
| 3 | OAS2, HERC5 and USP | 5.936e⁻⁷ |
| 4 | OAS2, HERC5, USP18 and UBE2L6 | 2.91e⁻⁷ |
| 5 | OAS2, HERC5, USP18, UBE2L6 and ISG15 | 4.73e⁻⁸ |
| 6 | OAS2, HERC5, USP18, UBE2L6, ISG15 and IFI27 | 2.71e⁻⁷ |
| 7 | OAS2, HERC5, USP18, UBE2L6, ISG15, IFI27 | 3.32e⁻⁷ |
| | and IFI44L | |
| 8 | OAS2, HERC5, USP18, UBE2L6, ISG15, IFI27, | 1.75e⁻⁶ |
| | IFI44L and UBE1L | |
| 9 | OAS2, HERC5, USP18, UBE2L6, ISG15, IFI27, | 5.53e⁻⁶ |
| | IFI44L, UBE1L and IFIH1 | |

As shown in Table 1 and FIG. 6, t-test analysis of each one of the summed expression described above, showed that the best prediction of a patient to be considered as responders or non-responder was obtained with a set of five genes OAS2, HERC5, USP18, UBE2L6 and ISG15.

The significance of the specific group of the five marker genes of the invention was further evaluated by examining different combinations thereof.

More specifically, the significant of other sets of five gens that include HERC5, USP18, UBE2L6 and ISG15 with an additional gene other than OAS2 was tested. The expression of the genes obtained from liver samples of HCV patients before initiation of treatment was determined, normalized and scales as described above.

The following combinations were tested for their ability to predict responsiveness to IFN treatment:
HERC5, USP18, UBE2L6, ISG15 and OAS2 (the set of above)
HERC5, USP18, UBE2L6, ISG15 and IFI44L
HERC5, USP18, UBE2L6, ISG15 and IFI27
HERC5, USP18, UBE2L6, ISG15 and IFIH1
HERC5, USP18, UBE2L6, ISG15 and UBE1L
HERC5, USP18, UBE2L6, ISG15 and FLJ42418
HERC5, USP18, UBE2L6, ISG15 and TLR7

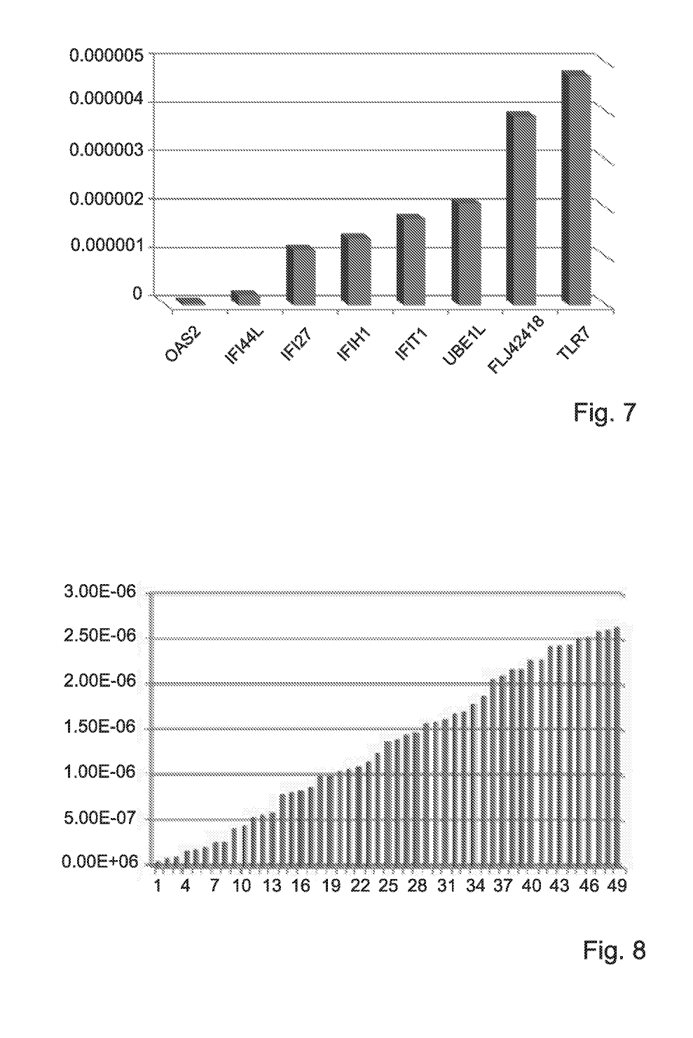

FIG. 7 shows the p-value obtained from t-test analysis and demonstrated that none of the tested combinations of five genes was better or even as good in predicting responsiveness as the combination described above, namely HERC5, USP18, UBE2L6, ISG15 and OAS2.

This shows the significance of the specific group of HERC5, USP18, UBE2L6, ISG15 and OAS2 in predicting responsiveness of a patient to IFN treatment.

In addition, different combinations of five genes from a group of 13 genes OAS2, HERC5, USP18, UBE2L6, ISG15, IFI27, IFI44L, UBE1L, IFIH1, p53, FLJ42418, TLR7 and IFITI were studied for their ability to predict responsiveness to treatment.

Table 2 shows the 50 groups of five genes that represent 50 permutations of the 13 genes noted above.

TABLE 2
list of combinations

| Group No. | 1^{st} gene | 2^{nd} gene | 3^{rd} gene | 4^{th} gene | 5^{th} gene |
|---|---|---|---|---|---|
| 1 | OAS2 | HERC5 | USP18 | UBE2L6 | ISG15 |
| 2 | OAS2 | HERC5 | USP18 | ISG15 | IF144L |
| 3 | OAS2 | USP18 | UBE2L6 | ISG15 | IF144L |
| 4 | OAS2 | HERC5 | USP18 | ISG15 | IFI27 |
| 5 | OAS2 | HERC5 | USP18 | UBE2L6 | IF144L |
| 6 | HERC5 | USP18 | UBE2L6 | ISG15 | IF144L |
| 7 | OAS2 | HERC5 | UBE2L6 | ISG15 | IF144L |
| 8 | OAS2 | HERC5 | USP18 | ISG15 | UBE1L |
| 9 | OAS2 | USP18 | UBE2L6 | ISG15 | IFI27 |
| 10 | OAS2 | HERC5 | USP18 | ISG15 | FLJ42418 |
| 11 | OAS2 | USP18 | ISG15 | IFI27 | IF144L |
| 12 | OAS2 | HERC5 | UBE2L6 | ISG15 | P53 |
| 13 | OAS2 | HERC5 | UBE2L6 | ISG15 | TLR7 |
| 14 | OAS2 | USP18 | ISG15 | IFI44L | UBE1L |
| 15 | OAS2 | USP18 | UBE2L6 | ISG15 | IFIH1 |
| 16 | OAS2 | HERC5 | USP18 | UBE2L6 | IFI27 |
| 17 | OAS2 | USP18 | UBE2L6 | ISG15 | UBE1L |
| 18 | OAS2 | USP18 | UBE2L6 | IFI27 | IF144L |
| 19 | OAS2 | HERC5 | USP18 | IFI27 | IF144L |
| 20 | OAS2 | USP18 | ISG15 | IFI44L | P53 |
| 21 | OAS2 | HERC5 | USP18 | ISG15 | IFIH1 |
| 22 | OAS2 | HERC5 | USP18 | IF144L | UBE1L |
| 23 | HERC5 | USP18 | UBE2L6 | ISG15 | IFI27 |
| 24 | OAS2 | HERC5 | USP18 | UBE2L6 | UBE1L |
| 25 | HERC5 | USP18 | UBE2L6 | ISG15 | IFIH1 |
| 26 | OAS2 | USP18 | UBE2L6 | IFI44L | UBE1L |
| 27 | OAS2 | HERC5 | USP18 | IFI44L | P53 |
| 28 | OAS2 | HERC5 | UBE2L6 | ISG15 | IFI27 |
| 29 | OAS2 | HERC5 | ISG15 | IF144L | P53 |
| 30 | HERC5 | USP18 | ISG15 | IFI27 | IFI44L |
| 31 | OAS2 | USP18 | UBE2L6 | ISG15 | IFIT1 |
| 32 | OAS2 | HERC5 | USP18 | UBE2L6 | FLJ42418 |
| 33 | OAS2 | USP18 | UBE2L6 | ISG15 | FLJ42418 |
| 34 | HERC5 | USP18 | UBE2L6 | ISG15 | IFIT1 |
| 35 | OAS2 | USP18 | UBE2L6 | ISG15 | TLR7 |
| 36 | OAS2 | HERC5 | USP18 | ISG15 | IFIT1 |
| 37 | HERC5 | USP18 | UBE2L6 | ISG15 | UBE1L |
| 38 | OAS2 | HERC5 | USP18 | UBE2L6 | P53 |
| 39 | HERC5 | USP18 | ISG15 | IFI44L | UBE1L |
| 40 | OAS2 | HERC5 | USP18 | IFI44L | FLJ42418 |
| 41 | OAS2 | HERC5 | UBE2L6 | ISG15 | UBE1L |
| 42 | OAS2 | USP18 | UBE2L6 | ISG15 | P53 |
| 43 | HERC5 | USP18 | UBE2L6 | IFI27 | IF144L |
| 44 | OAS2 | USP18 | ISG15 | IFI44L | TLR7 |
| 45 | OAS2 | HERC5 | UBE2L6 | ISG15 | IFIH1 |
| 46 | HERC5 | USP18 | ISG15 | IFI44L | P53 |
| 47 | OAS2 | USP18 | ISG15 | IFI44L | FLJ42418 |
| 48 | OAS2 | USP18 | ISG15 | IFI27 | UBE1L |
| 49 | OAS2 | HERC5 | USP18 | UBE2L6 | TLR7 |
| 50 | OAS2 | USP18 | ISG15 | IFI44L | IFIH1 |

FIG. 8 shows p-value obtained from t-test analysis of the 50 permutations detailed in Table 2. The results clearly indicated that the best and most significant group of all the tested permutations corresponds to the group of five genes HERC5, USP18, UBE2L6, ISG15 and OAS2.

The significance of the genetic data obtained before the treatment in HCV patients in evaluating prediction to treatment was evaluated by ROC curves. Specifically, the expression level of the five genes, OAS2, HERC5, USP18, UBE2L6 and ISG15 at day “0” was analyzed.

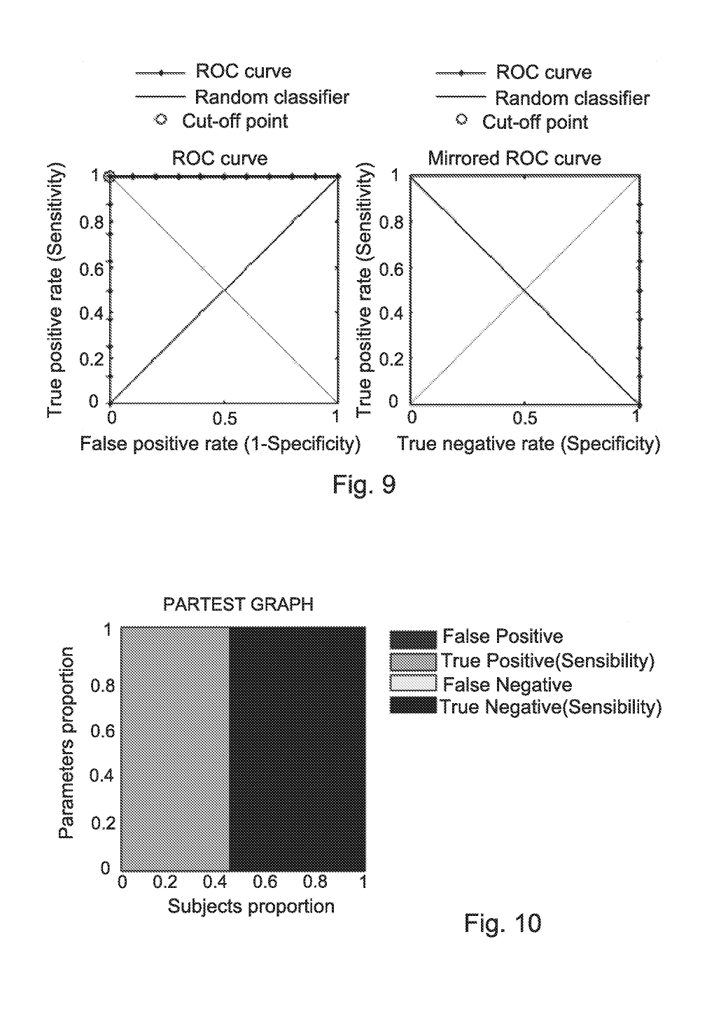

The ROC curve for evaluating the response to treatment in HCV patients before initiation of treatment using the genes OAS2, HERC5, USP18, UBE2L6 and ISG15 is shown in FIG. 9.

The diagnostic capability was provided by calculating the area under the Receiver Operating Characteristics (ROC) curves as described above. As can be seen for the purpose of diagnosis of response to treatment, the score using the five gens reached a very high area under the ROC curve of 100% until results are in 18 month.

Using this function, high positive predictive values and correct identification was obtained for 18 of the patients, out of 18 patients. The analysis showed sensitivity of 100% and a specificity of 100%.

The performance of the results was evaluated using partest method. The output is shown as graphical representation in FIG. 10 in partest graph.

All patients were true positive, namely were predicted to respond to treatment and were indeed found to be responsive. In addition, all patients were true negative, namely were predicted to not respond to treatment and were indeed not responders.

Based on these results, it can be clearly concluded that genetic data obtained before initiation of treatment from a HCV patient may accurately predict if the patient will respond to treatment based on the expression level of at least these five genes.

1-32. (canceled)

33. A kit comprising detecting molecules specific for determining the level of expression of OAS2, HERC5, USP18, UBE2L6 and optionally of ISG15, genes in a biological sample.

34. The kit according to claim 33, further comprising at least one of:
(a) detecting molecules specific for determining the level of expression of at least one control reference gene in a biological sample, wherein said detecting molecules comprise at least one of isolated detecting nucleic acid molecules and isolated detecting amino acid molecules;
(b) pre-determined calibration curve providing standard expression values of OAS2, HERC5, USP18, UBE2L6 and optionally of ISG15, genes;
(c) pre-determined calibration curve providing standard expression values of said at least one control reference gene; and
(d) at least one control sample.

35. The kit according to claim 33, wherein said kit is a prognostic kit for predicting and assessing responsiveness of a mammalian subject to interferon treatment.

36. The kit according to claim 35, wherein said subject is suffering from an infectious disease.

37. The kit according to claim 36, wherein said subject is suffering from a hepatitis C virus (HCV) infection.

38. The kit according to claim 33, further comprising instructions for use, wherein said instructions comprise at least one of:
(a) instructions for carrying out the detection and quantification of expression of said OAS2, HERC5, USP18, UBE2L6 and optionally of ISG15, genes;
(b) instructions for carrying out the detection and quantification of expression of said at least one control reference gene;
(c) instructions for determining if the expression values of said OAS2, HERC5, USP18, UBE2L6 and optionally of ISG15, genes is positive or negative with respect to a corresponding predetermined standard expression value of said genes.

39. The kit according to claim 33, wherein said detecting molecules comprise isolated oligonucleotides, each said oligonucleotide specifically hybridize to a nucleic acid sequence of an RNA product of one of said OAS2, HERC5, USP18, UBE2L6 and optionally of ISG15, genes, and wherein said detecting molecules are at least one of at least one primer, at least one pair of primers, at least one nucleotide probe and any combination thereof.

40. The kit according to claim 33, further comprising at least one reagent for conducting a nucleic acid amplification based assay selected from the group consisting of a Real-Time PCR, micro arrays, PCR, in situ Hybridization and Comparative Genomic Hybridization.

41. The kit according to claim 33, further comprising a solid support, wherein each of said detecting molecules is disposed in an array, and wherein:
(a) said array of detecting molecules comprises a plurality of addressed vessels; or
(b) said array of detecting molecules comprises a solid support holding detecting molecules in distinct regions.

42. The kit according to claim 33, wherein said sample is a blood sample, and wherein said kit comprises detecting molecule/s specific for determining the level of expression of OAS2, HERC5, USP18, UBE2L6 and optionally of ISG15, genes in said blood sample.

43. The kit according to claim 33, wherein said sample is a liver tissue biopsy sample, and wherein said kit comprises detecting molecule/s specific for determining the level of expression of OAS2, HERC5, USP18, UBE2L6 and ISG15 genes in said liver tissue biopsy sample.

44. An array of detecting molecules specific for OAS2, HERC5, USP18, UBE2L6 and optionally for ISG15, genes, wherein said detecting molecules are isolated detecting nucleic acid molecules or isolated detecting amino acid molecule/s, wherein said array optionally comprises:
(a) a plurality of addressed vessels containing said detecting molecule/s; or
(b) a solid support holding detecting molecules in distinct regions.

45. A prognostic composition comprising detecting molecules specific for determining the level of expression of OAS2, HERC5, USP18, UBE2L6 and optionally of ISG15, genes in a biological sample, wherein aid composition for predicting and assessing responsiveness of a mammalian subject to interferon treatment.

46. A prognostic method for predicting and assessing responsiveness of a mammalian subject to interferon treatment, said method comprising the steps of:
(a) determining the level of expression of OAS2, HERC5, USP18, UBE2L6 and optionally, of ISG15 genes in a biological sample of said subject to obtain an expression value;
(b) determining if the expression value obtained in step (a), is positive or negative with respect to a predetermined standard expression value or to an expression value of said OAS2, HERC5, USP18, UBE2L6 and optionally of ISG15, genes in at least one control sample;
wherein a positive expression value of said OAS2, HERC5, USP18, UBE2L6 and optionally of ISG15, genes as compared to said predetermined standard expression value or to said expression value of said genes in at least one control sample, indicates that said subject is not responsive to interferon treatment, thereby predicting responsiveness of a mammalian subject to interferon treatment.

47. The method according to claim 46, wherein determining the level of expression of said OAS2, HERC5, USP18, UBE2L6 and optionally of ISG15, genes in a biological sample of said subject is performed by the step of contacting detecting molecules specific for said genes with a biological sample of said subject, or with any nucleic acid or protein product obtained therefrom, and wherein said detecting molecules comprise at least one of isolated detecting nucleic acid molecule/s and isolated detecting amino acid molecules.

48. The method according to claim 47, wherein said nucleic acid detecting molecules comprise isolated oligonucleotides, each oligonucleotide specifically hybridizes to a nucleic acid sequence of a product of one of said OAS2, HERC5, USP18, UBE2L6 and optionally of ISG15, genes, and wherein said detecting molecules are at least one of at least one primer, at least one a pair of primers, at least one nucleotide probe and any combination thereof.

49. The method according to claim 46, wherein said subject is suffering from an infectious disease, and wherein said subject is suffering from an HCV infection.

50. The method according to claim 46, wherein said sample is a blood sample, and wherein said method comprises determining the level of expression of OAS2, HERC5, USP18, UBE2L6 and optionally of ISG15, genes in said blood sample.

51. The method according to claim 46, wherein said sample is a liver tissue biopsy sample, and wherein said method comprises determining the level of expression of OAS2, HERC5, USP18, UBE2L6 and ISG15 genes in said liver tissue sample.

52. The method according to claim 46, wherein said sample is obtained prior to interferon treatment of said subject.
